(19) United States
(12) Patent Application Publication
(10) Pub. No.: US 2024/0174986 A1
(43) Pub. Date: May 30, 2024

(54) MUTANT HERPESVIRUS AND VACCINE COMPOSITIONS

(71) Applicant: Rational Vaccines, Inc., Cambridge, MA (US)

(72) Inventors: Agustin FERNANDEZ, Cambridge, MA (US); Edward GERSHBURG, Cambridge, MA (US); Paul PREDKI, Cambridge, MA (US)

(73) Assignee: Rational Vaccines, Inc., Cambridge, MA (US)

(21) Appl. No.: 18/284,793

(22) PCT Filed: Mar. 28, 2022
(86) PCT No.: PCT/US2022/022211

§ 371 Date: Sep. 28, 2023

Related U.S. Application Data
(60) Provisional application No. 63/167,388, filed on Mar. 29, 2021.

(51) Int. Cl. C12N 7/00; A61K 39/00; A61K 39/245; A61P 31/22; C07K 14/005

(52) U.S. Cl. CPC C12N 7/00; A61K 39/245; A61P 31/22; C07K 14/005; A61K 2039/522; C12N 2710/16621; C12N 2710/16622; C12N 2710/16634

(57) ABSTRACT

Compositions and methods related to a mutant herpesvirus and a mutant ICP0 protein. Vaccine compositions comprising the mutant herpesvirus and/or mutant ICP0 protein. Methods of treatment using the mutant herpesvirus and/or mutant ICP0 protein.

### CROSS REFERENCE TO RELATED APPLICATIONS

This application claims priority to U.S. Provisional Application No. 63/167,388, filed on Mar. 29, 2021, which is incorporated by reference herein in its entirety.

### SEQUENCE LISTING

The Sequence Listing associated with this application is provided in text format in lieu of a paper copy and is hereby incorporated by reference into the specification. The name of the text file containing the Sequence Listing is RAVA-006-01WO_ST25.txt. The text file is 24 KB, was created on Mar. 28, 2022, and is being submitted electronically via EFS-Web.

### FIELD OF THE INVENTION

Embodiments of the disclosure concern at least the fields of molecular biology, cell biology, biochemistry, immunology, and medicine.

### BACKGROUND

Herpes simplex virus type 2 (HSV-2) is the leading cause of genital herpes and infects ˜1 billion people world-wide. HSV-2 is a DNA virus that often results in skin lesions and is characterized by latent and recurrent infections. HSV-2 can manifest as a cluster of small fluid-filled blisters that rupture and form painful sores, taking several weeks to heal, i.e., an outbreak. The virus can exist in nerve cells for the lifetime of the infected subject and reactivate at irregular intervals. Approximately 5% of HSV-2 infected individuals live with genital herpes disease that recurs once every 3-12 months. Even in the absence of actual ulcers, the virus can be produced and spread to new individuals at a rate of ˜20 million per year. Currently, there is no cure for HSV-2 infection.

Treatment options for HSV-2 symptoms are limited, and it is highly desirable to develop pharmaceutical compositions that inhibit or counteract infection by HSV-2. Live, attenuated vaccines are an attractive candidate. The ideal live, attenuated effective vaccine would be safe (avirulent), while eliciting a potent immune response in the subject (highly immunogenic). However, a safer and more attenuated live virus vaccine is often associated with a weaker immune response in the subject. Therefore, new and improved compositions of a herpesvirus vaccine that is highly immunogenic and essentially avirulent are desirable.

### SUMMARY

The present disclosure provides mutant herpesviruses and related compositions and methods of use.

In one aspect, the disclosure provides a mutant herpesvirus comprising a modified RL2 gene sequence, wherein the modified RL2 gene sequence encodes a polypeptide comprising one or more fragments of infected cell protein 0 (ICP0), wherein the polypeptide comprises or consists of, from the N-terminus to the C-terminus:
a first ICP0 sequence comprising a sequence having at least 80% sequence identity to SEQ ID NO: 1 and comprising no more than 30 amino acids;
optionally, a first non-ICP0 sequence;
a second ICP0 sequence comprising a sequence having at least 80% sequence identity to SEQ ID NO: 2 and comprising no more than 30 amino acids; and, optionally, a second non-ICP0 sequence;
wherein the first and second non-ICP0 sequences have less than 10%, less than 20%, less than 30%, less than 40%, or less than 50% sequence identity to ICP0, and wherein the mutant herpesvirus is substantially avirulent and immunogenic.

In certain embodiments, the herpesvirus is selected from herpes simplex virus 1 (HSV-1) or herpes simplex virus 2 (HSV-2). In certain embodiments, the polypeptide does not comprise an ICP0 region selected from a RING finger domain, a nuclear localization signal (NLS) domain, and an oligomerization domain. In certain embodiments, one or more of the RING finger domain, NLS domain and/or oligomerization domain of the mutant ICP0 polypeptide comprises a mutation, e.g., a deletion, such that the domain is not active or has reduced activity as compared to the wild type domain in the context of a native ICP0 polypeptide, e.g., activity is reduced by at least 10%, at least 20%, at least 30%, at least 40$, at least 50%, at least 60%, at least 70%, at least 80%, at least 90%, at least 95%, or at least 99%. In certain embodiments, the polypeptide does not comprise the first non-ICP0 sequence or the second non-ICP0 sequence. In certain embodiments, the polypeptide comprises a sequence having at least 80% sequence identity to SEQ ID NO: 7. In certain embodiments, the polypeptide has at least 80% sequence identity to SEQ ID NO: 7. In certain embodiments, the polypeptide comprises the second non-ICP0 sequence and does not comprise the first non-ICP0 sequence. In certain embodiments, the polypeptide comprises a sequence having at least 80% sequence identity to SEQ ID NO: 8. In certain embodiments, the polypeptide has at least 80% sequence identity to SEQ ID NO: 8. In certain embodiments, the polypeptide comprises the first non-ICP0 sequence and does not comprise the second non-ICP0 sequence. In certain embodiments, the polypeptide comprises a sequence having at least 80% sequence identity to SEQ ID NO: 9. In certain embodiments, the polypeptide has at least 80% sequence identity to SEQ ID NO: 9. In certain embodiments, the polypeptide comprises the first non-ICP0 sequence and the second non-ICP0 sequence. In certain embodiments, the first non-ICP0 sequence comprises at least 10, at least 20, at least 50, at least 100, or at least 200 amino acids, or between 10 and 200 amino acids. In certain embodiments, the first non-ICP0 sequence comprises at least 200 amino acids. In certain embodiments, the first non-ICP0 sequence comprises or consists of a green florescent protein (GFP) sequence. In certain embodiments, the first non-ICP0 sequence has at least 80% sequence identity to SEQ ID NO: 3. In certain embodiments, the second non-ICP0 sequence comprises at least 10, at least 20, at least 50, or at least 100 amino acids, or between 10 and 100 amino acids. In certain embodiments, the second non-ICP0 sequence comprises at least 100 amino acids. In certain embodiments, the second non-ICP0 sequence comprises a sequence having at least 80% sequence identity to SEQ ID NO: 4. In certain embodiments, the second non-ICP0 sequence has at least 80% sequence identity to SEQ ID NO: 4. In certain embodiments, the polypeptide comprises a sequence having at least 80% sequence identity to SEQ ID NO: 5. In certain embodiments, the polypeptide has at least 80% sequence identity to SEQ ID NO: 5.

In a related aspect, the disclosure provides an immunogenic composition comprising a pharmaceutically acceptable carrier and a mutant herpesvirus disclosed herein. In certain embodiments, the composition further comprises one or more adjuvant. In certain embodiments, the mutant herpesvirus comprises a modified RL2 gene sequence, wherein the modified RL2 gene sequence encodes a polypeptide comprising one or more fragments of infected cell protein 0 (ICP0), wherein the polypeptide comprises or consists of, from the N-terminus to the C-terminus:
a first ICP0 sequence comprising a sequence having at least 80% sequence identity to SEQ ID NO: 1 and comprising no more than 30 amino acids;
optionally, a first non-ICP0 sequence;
a second ICP0 sequence comprising a sequence having at least 80% sequence identity to SEQ ID NO: 2 and comprising no more than 30 amino acids; and,
optionally, a second non-ICP0 sequence;
wherein the first and second non-ICP0 sequences have less than 10%, less than 20%, less than 30%, less than 40%, or less than 50% sequence identity to ICP0, and wherein the mutant herpesvirus is substantially avirulent and immunogenic.

In certain embodiments of the composition, the herpesvirus is selected from herpes simplex virus 1 (HSV-1) or herpes simplex virus 2 (HSV-2). In certain embodiments, the polypeptide does not comprise an ICP0 region selected from a RING finger domain, a nuclear localization signal (NLS) domain, and an oligomerization domain. In certain embodiments, the polypeptide does not comprise the first non-ICP0 sequence or the second non-ICP0 sequence. In certain embodiments, the polypeptide comprises a sequence having at least 80% sequence identity to SEQ ID NO: 7. In certain embodiments, the polypeptide has at least 80% sequence identity to SEQ ID NO: 7. In certain embodiments, the polypeptide comprises the second non-ICP0 sequence and does not comprise the first non-ICP0 sequence. In certain embodiments, the polypeptide comprises a sequence having at least 80% sequence identity to SEQ ID NO: 8. In certain embodiments, the polypeptide has at least 80% sequence identity to SEQ ID NO: 8. In certain embodiments, the polypeptide comprises the first non-ICP0 sequence and does not comprise the second non-ICP0 sequence. In certain embodiments, the polypeptide comprises a sequence having at least 80% sequence identity to SEQ ID NO: 9. In certain embodiments, the polypeptide has at least 80% sequence identity to SEQ ID NO: 9. In certain embodiments, the polypeptide comprises the first non-ICP0 sequence and the second non-ICP0 sequence. In certain embodiments, the first non-ICP0 sequence comprises at least 10, at least 20, at least 50, at least 100, or at least 200 amino acids, or between 10 and 200 amino acids. In certain embodiments, the first non-ICP0 sequence comprises at least 200 amino acids. In certain embodiments, the first non-ICP0 sequence comprises or consists of a green florescent protein (GFP) sequence. In certain embodiments, the first non-ICP0 sequence comprises a sequence having at least 80% sequence identity to SEQ ID NO: 3. In certain embodiments, the first non-ICP0 sequence has at least 80% sequence identity to SEQ ID NO: 3. In certain embodiments, the second non-ICP0 sequence comprises at least 10, at least 20, at least 50, or at least 100 amino acids, or between 10 and 100 amino acids. In certain embodiments, the second non-ICP0 sequence comprises at least 100 amino acids. In certain embodiments, the second non-ICP0 sequence comprises a sequence having at least 80% sequence identity to SEQ ID NO: 4. In certain embodiments, the second non-ICP0 sequence has at least 80% sequence identity to SEQ ID NO: 4. In certain embodiments, the polypeptide comprises a sequence having at least 80% sequence identity to SEQ ID NO: 5. In certain embodiments, the polypeptide has at least 80% sequence identity to SEQ ID NO: 5.

In a further related aspect, the disclosure provides method of immunizing a subject, the method comprising administering to the subject a mutant herpesvirus or immunogenic composition disclosed herein. In certain embodiments, the subject is human. In certain embodiments, the mutant herpesvirus or immunogenic composition is administered to the subject in a therapeutically effective amount to induce an immunological response to the mutant herpesvirus. In certain embodiments, the method further comprises administering to the subject a booster dose of the mutant herpes virus or immunogenic composition. In certain embodiments, the method is practiced for treating a herpesvirus infection in the subject. In certain embodiments, the method is practiced for preventing or inhibiting a herpesvirus infection in the subject. In certain embodiments, the herpesvirus infection is oral herpes. In certain embodiments, the herpesvirus infection is genital herpes. In certain embodiments, the mutant herpesvirus comprises a modified RL2 gene sequence, wherein the modified RL2 gene sequence encodes a polypeptide comprising one or more fragments of infected cell protein 0 (ICP0), wherein the polypeptide comprises or consists of, from the N-terminus to the C-terminus:
a first ICP0 sequence comprising a sequence having at least 80% sequence identity to SEQ ID NO: 1 and comprising no more than 30 amino acids;
optionally, a first non-ICP0 sequence;
a second ICP0 sequence comprising a sequence having at least 80% sequence identity to SEQ ID NO: 2 and comprising no more than 30 amino acids; and,
optionally, a second non-ICP0 sequence;
wherein the first and second non-ICP0 sequences have less than 10% sequence identity to ICP0, and
wherein the mutant herpesvirus is substantially avirulent and immunogenic.

In certain embodiments of the methods disclosed, the herpesvirus is selected from herpes simplex virus 1 (HSV-1) or herpes simplex virus 2 (HSV-2). In certain embodiments, the polypeptide does not comprise an ICP0 region selected from a RING finger domain, a nuclear localization signal (NLS) domain, and an oligomerization domain. In certain embodiments, the polypeptide does not comprise the first non-ICP0 sequence or the second non-ICP0 sequence. In certain embodiments, the polypeptide comprises a sequence having at least 80% sequence identity to SEQ ID NO: 7. In certain embodiments, the polypeptide has at least 80% sequence identity to SEQ ID NO: 7. In certain embodiments, the polypeptide comprises the second non-ICP0 sequence and does not comprise the first non-ICP0 sequence. In certain embodiments, the polypeptide comprises a sequence having at least 80% sequence identity to SEQ ID NO: 8. In certain embodiments, the polypeptide has at least 80% sequence identity to SEQ ID NO: 8. In certain embodiments, the polypeptide comprises the first non-ICP0 sequence and does not comprise the second non-ICP0 sequence. In certain embodiments, the polypeptide comprises a sequence having at least 80% sequence identity to SEQ ID NO: 9. In certain embodiments, the polypeptide has at least 80% sequence identity to SEQ ID NO: 9. In certain embodiments, the polypeptide comprises the first non-ICP0 sequence and the second non-ICP0 sequence. In certain embodiments, the first non-ICP0 sequence comprises at least 10, at least 20, at least 50, at least 100, or at least 200 amino acids, or between 10 and 200 amino acids. In certain embodiments, the first non-ICP0 sequence comprises at least 200 amino acids. In certain embodiments, the first non-ICP0 sequence comprises or consists of a green florescent protein (GFP) sequence. In certain embodiments, the first non-ICP0 sequence comprises a sequence having at least 80% sequence identity to SEQ ID NO: 3. In certain embodiments, the first non-ICP0 sequence has at least 80% sequence identity to SEQ ID NO: 3. In certain embodiments, the second non-ICP0 sequence comprises at least 10, at least 20, at least 50, or at least 100 amino acids, or between 10 and 100 amino acids. In certain embodiments, the second non-ICP0 sequence comprises at least 100 amino acids. In certain embodiments, the second non-ICP0 sequence comprises a sequence having at least 80% sequence identity to SEQ ID NO: 4. In certain embodiments, the second non-ICP0 sequence has at least 80% sequence identity to SEQ ID NO: 4. In certain embodiments, the polypeptide comprises a sequence having at least 80% sequence identity to SEQ ID NO: 5. In certain embodiments, the polypeptide has at least 80% sequence identity to SEQ ID NO: 5.

In another related aspect, the disclosure provides a mutant ICP0 polypeptide comprising or consisting of, from the N-terminus to the C-terminus:
a. a first ICP0 sequence comprising a sequence having at least 80% sequence identity to SEQ ID NO: 1 and comprising no more than 30 amino acids;
b. optionally, a first non-ICP0 sequence;
c. a second ICP0 sequence comprising a sequence having at least 80% sequence identity to SEQ ID NO: 2 and comprising no more than 30 amino acids; and
d. optionally, a second non-ICP0 sequence;
wherein the first and second non-ICP0 sequences have less than 10% sequence identity to ICP0.

In certain embodiments, the herpesvirus is selected from herpes simplex virus 1 (HSV-1) or herpes simplex virus 2 (HSV-2). In certain embodiments, the polypeptide does not comprise an ICP0 region selected from a RING finger domain, a nuclear localization signal (NLS) domain, and an oligomerization domain. In certain embodiments, the polypeptide does not comprise the first non-ICP0 sequence or the second non-ICP0 sequence. In certain embodiments, the polypeptide comprises a sequence having at least 80% sequence identity to SEQ ID NO: 7. In certain embodiments, the polypeptide has at least 80% sequence identity to SEQ ID NO: 7. In certain embodiments, the polypeptide comprises the second non-ICP0 sequence and does not comprise the first non-ICP0 sequence. In certain embodiments, the polypeptide comprises a sequence having at least 80% sequence identity to SEQ ID NO: 8. In certain embodiments, the polypeptide has at least 80% sequence identity to SEQ ID NO: 8. In certain embodiments, the polypeptide comprises the first non-ICP0 sequence and does not comprise the second non-ICP0 sequence. In certain embodiments, the polypeptide comprises a sequence having at least 80% sequence identity to SEQ ID NO: 9. In certain embodiments, the polypeptide has at least 80% sequence identity to SEQ ID NO: 9. In certain embodiments, the polypeptide comprises the first non-ICP0 sequence and the second non-ICP0 sequence. In certain embodiments, the first non-ICP0 sequence comprises at least 10, at least 20, at least 50, at least 100, or at least 200 amino acids, or between 10 and 200 amino acids. In certain embodiments, the first non-ICP0 sequence comprises at least 200 amino acids. In certain embodiments, the first non-ICP0 sequence comprises or consists of a green florescent protein (GFP) sequence. In certain embodiments, the first non-ICP0 sequence comprises a sequence having at least 80% sequence identity to SEQ ID NO: 3. In certain embodiments, the first non-ICP0 sequence has at least 80% sequence identity to SEQ ID NO: 3. In certain embodiments, the second non-ICP0 sequence comprises at least 10, at least 20, at least 50, or at least 100 amino acids, or between 10 and 100 amino acids. In certain embodiments, the second non-ICP0 sequence comprises at least 100 amino acids. In certain embodiments, the second non-ICP0 sequence comprises a sequence having at least 80% sequence identity to SEQ ID NO: 4 In certain embodiments, the second non-ICP0 sequence has at least 80% sequence identity to SEQ ID NO: 4. In certain embodiments, the polypeptide comprises a sequence having at least 80% sequence identity to SEQ ID NO: 5. In certain embodiments, the polypeptide has at least 80% sequence identity to SEQ ID NO: 5.

In another aspect, the disclosure provides a polynucleotide comprising a sequence encoding a mutant ICP0 polypeptide disclosed herein.

In another aspect, the disclosure provides a cell comprising a mutant ICP0 polypeptide disclosed herein, a mutant herpesvirus disclosed herein, or a polynucleotide disclosed herein.

### BRIEF DESCRIPTION OF THE DRAWINGS

Various objects and advantages and a more complete understanding of the disclosure are apparent and more readily appreciated by reference to the following Detailed Description and to the appended claims when taken in conjunction with the accompanying Drawing wherein:

FIG. 1 illustrates a schematic diagram of the wild type HSV-2 ICP0 gene (panel A) and the corresponding wild type HSV-2 ICP0 protein (panel B). The relative location of the ICP0 genes in the genome is shown in panel A. The three conserved domains, the Ring finger domain, NLS domain, and oligomerization domain, are annotated in panel B. Homology of the three conserved regions between HSV-1 and HSV-2 is also shown in panel B.

FIG. 2 illustrates a schematic diagram of a mutant ICP0 polypeptide (panel C) compared to wild type ICP0 (panel A) and ICP0 with a GPFΔ19-104 mutation (panel B). In this embodiment, the mutant ICP0 comprises a small region at the N terminus of the wild type protein that is separated by a GFP protein. Optionally, an additional non-ICP0 sequence may be inserted at the C terminus of the mutant ICP0.

FIG. 3 is a graph showing survival at various times post-challenge with HSV-2. Mice in Groups 2-5 were challenged via intra-vaginal inoculation with HSV-2 virus, 90 days post primary vaccination and observed for 30 days. One hundred percent (100%) of the test article vaccinated animals (Group 3, 4 and Group 5) survived the HSV-2 challenge. A highly statistically significant difference (p<0.0001) was observed between the vaccinated (Groups 3, 4, 5) and mock-vaccinated group (Group 2) using the Gehan-Breslow-Wilcoxin test.

FIG. 4 is a graph showing body weight at various times before and post-challenge with HSV-2 on day 90.

FIG. 5 is a graph showing the results of a neutralization assay, indicating fold increase in titer over 120 days with treatment with low dose, mid dose or high dose vaccination. The lines from top to bottom at day 90 correspond to: low dose, mid dose, and high dose, respectively.

### DETAILED DESCRIPTION

The present invention is described more fully hereinafter using illustrative, non-limiting embodiments, and references to the accompanying figures. This invention may, however, be embodied in many different forms and should not be construed as to be limited to the embodiments set forth below. Rather, these embodiments are provided so that this disclosure is thorough and conveys the scope of the invention to those skilled in the art.

Herpes simplex virus (HSV) type 1 (HSV-1) or type 2 (HSV-2) include an infected cell protein 0 (ICP0) as an immediate-early (IE) transactivator and E3 ubiquitin (Ub) ligase, which disrupts nuclear domain 10 (ND10) and inhibits the cellular interferon response. Cellular functions of ICP0 are involved in viral gene expression, viral replication, and reactivation from latency. ICP0 is a nuclear protein that is phosphorylated, a post-translational modification that acts as a key regulator of many viral and cellular proteins. ICP0 is the product of gene IE-1 (or α0) for HSV-1 and the product of gene RL2 for HSV-2. Illustrative wild-type IPCO sequences for HSV-1 strain KOS, HSV-1 strain Syn17+, and HSV-2 strain HG52 are provided in U.S. Pat. No. 8,802,109. An illustrative HSV-2 strain MS ICP0 polypeptide sequence is shown below:

| (SEQ ID NO: 11) | |
|---|---|
| 1 | meprpgtssr adpgperppr qtpgtqpaap hawgmlndmq wlassdseee tevgisdddl |
| 61 | hrdstseags tdtemfeagl mdaatpparp paerqgsptp adaqgscggg pvgeeeaeag |
| 121 | gggdvcavct deiapplrcq sfpclhpfci pcmktwiplr ntcplentpv aylivgvtas |
| 181 | gsfstipivn dprtrveaea avrsgtavdf iwtgnprtap rslslgghtv ralsptppwp |
| 241 | gtddedddla dadyvppapr raprrgggga gatrgtsqpa atrpappgap rssssggapl |
| 301 | ragvgsgsgg gpavaavvpr vaslppaagg graqarrvge daaaaegrtp parqpraaqe |
| 361 | ppivisdspp psprrpagpg plsffssssa qvssgpgggg lpqssgraar praavaprvr |
| 421 | sppraaaapv vsasadaagp appavpvdah raprsrmtqa qtdtqaqslg ragatdargs |
| 481 | ggpgaeggpg vprgtntpga aphaaegaaa rprkrrgsds gpaasssass saaprsplap |
| 541 | qgvgakraap rrapdsdsgd rghgplapas agaappsasp ssqaavaaaa sssssssssa |
| 601 | sssaggaggs vasasgager retslgpraa aprgprkcar ktrhaeggpe pgardpapgl |
| 661 | trylpiagas svvalapyvn ktvtgdclpi ldmetghiga yvvlvdqtgn vadllraaap |
| 721 | awsrrtllpe harncvrppd yptppasewn slwmtpvgnm lfdqgtlvga ldfhglrsrh |
| 781 | pwsreqgapa pagdapaghg e. |

### Conserved ICP0 Regions

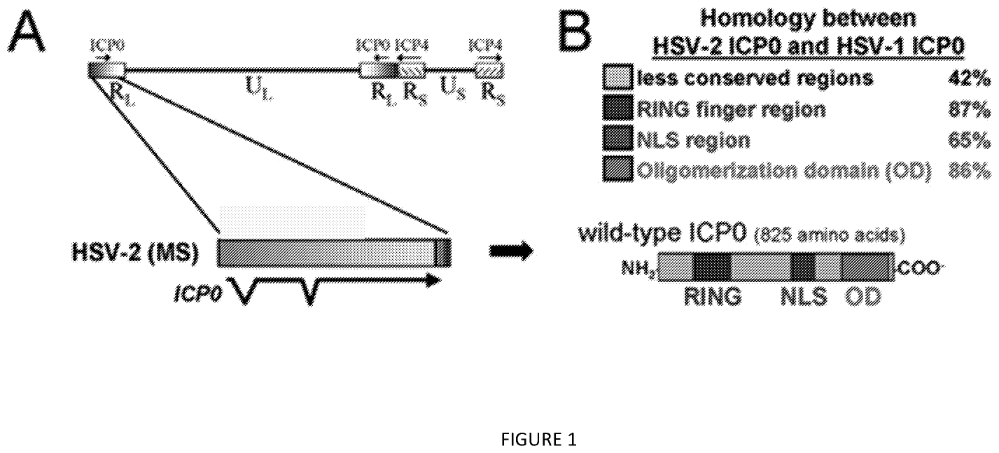

Three regions of ICP0 are highly conserved between HSV-1 and HSV-2, which are thought to have diverged from a common ancestral virus millions of years ago. These conserved regions include the N-terminal RING finger domain, the nuclear localization signal (NLS) domain near the middle of the protein, and C-terminal oligomerization domain (also known as the multimerization domain) (FIG. 1). Studies have shown that mutations in any one of the RING finger domain, NLS domain, and oligomerization domain of ICP0 (the three conserved domains) may render the herpesvirus avirulent, interferon-sensitive, but still highly immunogenic. However, HSV-2 mutants lacking all three conserved domains of ICP0 were found to be weakly immunogenic and less effective as a vaccine composition compared to HSV-2 mutants lacking only one conserved domain (U.S. Pat. No. 8,802,109). Inventors of the disclosure surprisingly discovered that certain HSV mutants lacking all three conserved domains are still highly immunogenic with decreased virulence compared to HSV mutants lacking only one conserved domain, making them more desirable candidates as vaccine compositions.

The term “mutation” as used in the disclosure may include alterations to an amino acid sequence or nucleotide sequence, including but not limited to, insertions, deletions, substitutions, and rearrangements. In certain embodiments, a mutation renders the mutant ICP0 or the mutated domain non-functional or having reduced functional activity, e.g., activity is reduced by at least 10%, at least 20%, at least 30%, at least 40%, at least 50%, at least 60%, at least 70%, at least 80%, at least 90%, at least 95%, or at least 99%.

The N-terminal RING finger domain is about 87% conserved in amino acid homology between HSV-1 and HSV-2, occurring between amino acid 113 and 242 of the HSV-1 ICP0 and between amino acid 123 and 252 of the HSV-2 ICP0. As used in the disclosure, the HSV-1 ICP0 RING finger domain may be referred to as RING-1, and the HSV-2 ICP0 RING finger domain may be referred to as RING-2. In some embodiments, the disclosure provides a mutant herpesvirus comprising a mutant ICP0 protein comprising a mutation in the RING finger domain. In some embodiments, the disclosure provides a mutant HSV-1 comprising a mutant ICP0 protein comprising a mutation in the RING-1 domain. In some embodiments, the disclosure provides a mutant HSV-2 comprising a mutant ICP0 protein comprising a mutation in the RING-2 domain.

The NLS domain is about 65% conserved in amino acid homology between HSV-1 and HSV-2, occurring between amino acid 453 and 531 of the HSV-1 ICP0 and between amino acid 468 and 549 of the HSV-2 ICP0. As used in the disclosure, the HSV-1 ICP0 NLS domain may be referred to as NLS-1, and the HSV-2 ICP0 NLS domain may be referred to as NLS-2. In some embodiments, the disclosure provides a mutant herpesvirus comprising a mutant ICP0 protein comprising a mutation in the NLS domain. In some embodiments, the disclosure provides a mutant HSV-1 comprising a mutant ICP0 protein comprising a mutation in the NLS-1 domain. In some embodiments, the disclosure provides a mutant HSV-2 comprising a mutant ICP0 protein comprising a mutation in the NLS-2 domain.

The oligomerization domain is about 86% conserved in amino acid homology between HSV-1 and HSV-2, occurring between amino acid 604 and 765 of the HSV-1 ICP0 and between amino acid 646 and 810 of the HSV-2 ICP0. As used in the disclosure, the HSV-1 ICP0 oligomerization domain may be referred to as OD-1, and the HSV-2 ICP0 oligomerization domain may be referred to as OD-2. In some embodiments, the disclosure provides a mutant herpesvirus comprising a mutant ICP0 protein comprising a mutation in the oligomerization domain. In some embodiments, the disclosure provides a mutant HSV-1 comprising a mutant ICP0 protein comprising a mutation in the OD-1 domain. In some embodiments, the disclosure provides a mutant HSV-2 comprising a mutant ICP0 protein comprising a mutation in the OD-2 domain.

In some embodiments, the disclosure provides a mutant herpesvirus comprising a mutant ICP0 protein comprising a mutation in at least one of the Ring finger domain, the NLS domain, and the oligomerization domain. In some embodiments, the disclosure provides a mutant herpesvirus comprising a mutant ICP0 protein that lacks the Ring finger domain, the NLS domain, and the oligomerization domain.

Within the conserved regions of ICP0, such as the Ring finger domain and NLS domain, there are phosphorylation sites that can alter DNA replication activity, affecting the biochemical and biological functions of the herpesvirus. A link exists between ICP0 's post translational modification state and HSV transactivating activity, which includes posttranslational phosphorylation. An ICP0 that is mutated may reduce or inhibit posttranslational phosphorylation and may decrease virulence in the mutant virus. Mutations in phosphorylation sites may also result in the virus encoding for the mutation to have inhibited replication. As used in the disclosure, the term “avirulent” may refer to a mutant virus that is unable to cause disease or significantly inhibited from causing disease. For example, an avirulent virus may be considered to be replication-impaired, repression-prone, and/or interferon-sensitive. As used in the disclosure, the term “immunogenic” may refer to a mutant virus that is capable of causing or inducing an immune response. For example, an immunogenic virus may be useful in an immunogenic composition, such as a vaccine.

### Mutant ICP0 Polypeptides

In some embodiments, the disclosure provides a mutant ICP0 polypeptide. In some embodiments, the mutant ICP0 polypeptide can inhibit replication of a herpesvirus (e.g., HSV-1 or HSV-2) comprising said mutant ICP0 polypeptide instead of a wild type ICP0 polypeptide. In some embodiments, a herpesvirus comprising said mutant ICP0 polypeptide is substantially avirulent and immunogenic, e.g., as compared to the same herpesvirus but comprising the wild type ICP0 polypeptide.

In some embodiments, a herpesvirus comprising said mutant ICP0 has impaired virus replication and/or delayed expression of early and late gene transcripts, e.g., as compared to the same herpesvirus but comprising the wild type ICP0 polypeptide. In certain embodiments, viral replication and/or expression of early and late gene transcripts is reduced or delayed by at least 10%, at least 20%, at least 30%, at least 40%, at least 50%, at least 60%, at least 70%, at least 80%, or at least 90% as compared to the same herpesvirus but comprising the wild type ICP0 polypeptide. In particular embodiments, the wild type ICP0 polypeptide is set forth in SEQ ID NO: 11.

In some embodiments, the mutant ICP0 polypeptide may be used as a prophylactic and/or therapeutic treatment with respect to herpesvirus infections or other diseases associated with herpesvirus infection.

In some embodiments, the disclosure provides a mutant ICP0 polypeptide comprising or consisting of one or more fragments of ICP0, wherein the polypeptide comprises or consists of, from the N-terminus to the C-terminus: a first ICP0 sequence comprising a sequence having at least 80%, at least 85%, at least 90%, at least 95%, at least 98%, at least 99%, or 100% sequence identity to SEQ ID NO: 1 and comprising no more than 30 amino acids: a first non-ICP0 sequence: a second ICP0 sequence comprising a sequence having at least 80%, at least 85%, at least 90%, at least 95%, at least 98%, at least 99%, or 100% sequence identity to SEQ ID NO: 2 and comprising no more than 30 amino acids; and, optionally, a second non-ICP0 sequence; wherein the first and second non-ICP0 sequences have less than 10% sequence identity to ICP0. In various embodiments, a non-ICP0 sequence has less than 50%, less than 40%, less than 30%, less than 20%, or less than 10% sequence identity to a region of wild type ICP0 of the same length. In particular embodiments, the wild type ICP0 polypeptide is set forth in SEQ ID NO: 11.

In some embodiments, the mutant ICP0 polypeptide lacks an ICP0 region selected from a RING finger region, an NLS region, and an oligomerization domain. In some embodiments of the mutant ICP0 polypeptide of the disclosure, the polypeptide lacks two ICP0 regions selected from a RING finger region, an NLS region, and an oligomerization domain. In some embodiments of the mutant ICP0 polypeptide of the disclosure, the polypeptide lacks a RING finger region, an NLS region, and an oligomerization domain.

In some embodiments of the mutant ICP0 polypeptide of the disclosure, the first non-ICP0 sequence comprises at least 50, at least 100, at least 150, or at least 200 amino acids. In some embodiments of the mutant ICP0 polypeptide of the disclosure, the first non-ICP0 sequence comprises at least 200 amino acids. In some embodiments of the mutant ICP0 polypeptide of the disclosure, the first non-ICP0 sequence comprises about 235 amino acids. In some embodiments of the mutant ICP0 polypeptide of the disclosure, the first non-ICP0 sequence comprises less than 1000, less than 800, less than 700, less than 600, less than 500, less than 400, or less than 300 amino acids. In some embodiments of the mutant ICP0 polypeptide of the disclosure, the first non-ICP0 sequence comprises 50-1000, 50-800, 50-600, 50-500, 50-300, 100-300, 200-300, or about 235 amino acids.

In some embodiments of the mutant ICP0 polypeptides of the disclosure, the mutant ICP0 polypeptide comprises or consists of a polypeptide sequence having at least 80%, at least 85%, at least 90%, at least 95%, at least 98%, at least 99%, or 100% sequence identity to any one of SEQ ID NOs: 1-5 or 7-10. In certain embodiments, it comprises or consists of SEQ ID NO: 1 and SEQ ID NO: 2, or SEQ ID NO:5.

In some embodiments of the mutant ICP0 polypeptide of the disclosure, the first non-ICP0 sequence comprises or consists of a florescent protein sequence. In some embodiments of the mutant ICP0 polypeptide of the disclosure, the first non-ICP0 sequence comprises or consists of a green florescent protein (GFP) sequence. In some embodiments of the mutant ICP0 polypeptide of the disclosure, the first non-ICP0 sequence has at least 80%, at least 85%, at least 90%, at least 95%, at least 98%, at least 99%, or 100% sequence identity to SEQ ID NO: 3.

In some embodiments of the mutant ICP0 polypeptide of the disclosure, the mutant ICP0 comprises or consists of the first ICP0 sequence and the second ICP0 sequence but does not comprise the first non-ICP0 sequence or the second non-ICP0 sequence. For example, in some embodiments, the mutant ICP0 may have at least 80%, at least 85%, at least 90%, at least 95%, at least 98%, at least 99%, or 100% sequence identity to SEQ ID NO: 7.

In some embodiments of the mutant ICP0 polypeptide of the disclosure, the mutant ICP0 comprises or consists of the second non-ICP0 sequence but not the first non-ICP0 sequence. For example, in some embodiments, the mutant ICP0 may have at least 80%, at least 85%, at least 90%, at least 95%, at least 98%, at least 99%, or 100% sequence identity to SEQ ID NO: 8.

In some embodiments of the mutant ICP0 polypeptide of the disclosure, the mutant ICP0 comprises or consists of the first non-ICP0 sequence but not the second non-ICP0 sequence. For example, in some embodiments, the mutant ICP0 may have at least 80%, at least 85%, at least 90%, at least 95%, at least 98%, at least 99%, or 100% sequence identity to SEQ ID NO: 9.

In some embodiments of the mutant ICP0 polypeptide of the disclosure, the mutant ICP0 comprises or consist of both the first non-ICP0 sequence and the second non-ICP0 sequence. For example, in some embodiments, the mutant ICP0 may have at least 80%, at least 85%, at least 90%, at least 95%, at least 98%, at least 99%, or 100% sequence identity to SEQ ID NO: 5.

In some embodiments of the mutant ICP0 polypeptide of the disclosure, the second non-ICP0 sequence comprises at least 50, at least 100, at least 150, or at least 200 amino acids. In some embodiments of the mutant ICP0 polypeptide of the disclosure, the second non-ICP0 sequence comprises at least 100 amino acids. In some embodiments of the mutant ICP0 polypeptide of the disclosure, the second non-ICP0 sequence comprises about 124 amino acids. In some embodiments of the mutant ICP0 polypeptide of the disclosure, the second non-ICP0 sequence comprises less than 100, less than 150, less than 200, less than 300, less than 400, or less than 500 amino acids. In some embodiments of the mutant ICP0 polypeptide of the disclosure, the second non-ICP0 sequence comprises 50-500, 50-400, 50-300, 50-200, 50-150, 75-150, 100-150, or about 125 amino acids.

In some embodiments of the mutant ICP0 polypeptide of the disclosure, the second non-ICP0 sequence is a randomly generated amino acid sequence. In some embodiments of the mutant ICP0 polypeptide of the disclosure, the second non-ICP0 sequence has at least at least 80%, at least 85%, at least 90%, at least 95%, at least 98%, at least 99%, or 100% sequence identity to SEQ ID NO: 4. In some embodiments of the mutant ICP0 polypeptide of the disclosure, the mutant ICP0 polypeptide lacks the second non-ICP0 sequence.

In some embodiments, the disclosure provides a polynucleotide sequence, such as a plasmid or gene or RNA, which encodes for the mutant ICP0 polypeptide of the disclosure.

In some embodiments, the disclosure provides a polynucleotide sequence encoding a mutant ICP0 polypeptide comprising one or more fragments of ICP0, wherein the polypeptide comprises or consists of, from the N-terminus to the C-terminus: a first ICP0 sequence comprising a sequence having at least 80% sequence identity to SEQ ID NO: 1 and comprising no more than 30 amino acids: a first non-ICP0 sequence: a second ICP0 sequence comprising a sequence having at least 80% sequence identity to SEQ ID NO: 2 and comprising no more than 30 amino acids; and, optionally, a second non-ICP0 sequence; wherein the first and second non-ICP0 sequences have less than 10% sequence identity to ICP0.

In some embodiments of the polynucleotide sequence encoding a mutant ICP0 polypeptide of the disclosure, the polypeptide lacks an ICP0 region selected from a RING finger region, an NLS region, and an oligomerization domain. In some embodiments of the polynucleotide sequence encoding a mutant ICP0 polypeptide of the disclosure, the polypeptide lacks two ICP0 regions selected from a RING finger region, an NLS region, and an oligomerization domain. In some embodiments of the polynucleotide sequence encoding a mutant ICP0 polypeptide of the disclosure, the polypeptide lacks a RING finger region, an NLS region, and an oligomerization domain.

An illustrative example of a polynucleotide sequence of the disclosure is provided in SEQ ID NO: 6.

### Mutant Herpesvirus

In some embodiments, the disclosure provides a mutant herpesvirus comprising one or more mutations in ICP0 that affects the virus's ability to replicate, establish latency, and reactivate from latency. The mutant herpesvirus may comprise a mutant gene that encodes for the mutant ICP0. In particular embodiments, the mutant ICP0 is any of the mutant or modified ICP0 disclosed herein, or a variant or fragment thereof. In some embodiments, the disclosure provides a mutant herpesvirus comprising mutations in ICP0 that is sensitive to cellular antiviral factors, such as interferon. In some embodiments, the disclosure provides a mutant herpesvirus comprising mutations in ICP0, wherein the mutant herpesvirus is avirulent, has inhibited replication and inhibited reactivation in comparison with wild-type herpesvirus, and/or is immunogenic. In some embodiments, the disclosure provides a mutant herpesvirus comprising mutations in ICP0, wherein the herpesvirus has inhibited or reduced replication or reactivation in both peripheral and central nervous systems. Such a mutant herpesvirus with a mutated ICP0 may protect a susceptible subject immunized therewith against infection by the corresponding wild-type herpesvirus. In some embodiments, the mutant herpesvirus encoding for a mutant ICP0 protein may be used in an immunogenic composition (e.g., vaccine composition or pharmaceutical composition) to inhibit replication of the corresponding wild-type herpesvirus, thereby providing treatment and/or prevention of diseases caused by the wild-type herpesvirus. In some embodiments, the mutant herpesvirus of the disclosure can inhibit replication of the corresponding wild type herpesvirus.

In some embodiments, the disclosure provides a mutant herpesvirus selected from herpes simplex virus 1 (HSV-1) or herpes simplex virus 2 (HSV-2). In certain embodiments, the mutant HSV-2 is the MS strain or based on the MS strain. In some embodiments, the disclosure provides a mutant herpesvirus selected from an α-herpesviruses comprising an ICP0-like protein. Illustrative examples of said α-herpesvirus include, but are not limited to, bovine herpesviruses 1 or 5 (BHV-1 or BHV-5), equid herpesviruses 1, 4, or 9 (EHV-1, EHV-4, or EHV-9), suid herpesvirus 1 or pseudorabiesvirus (PRV), varicella Zoster virus (VZV), canid herpesvirus 1 (CHV-1), felid herpesvirus 1 (FHV-1), macropodid herpesvirus 1 (MHV-1), cercopithecine herpesviruses 2 or 9 (CpHV-2 or CpHV-9), macacine herpesvirus 1 (commonly known as the herpes B virus), and papiline herpesvirus 2 (PHV-2).

In some embodiments, the disclosure provides a mutant herpesvirus comprising a modified RL2 gene sequence, wherein the modified RL2 gene sequence encodes a polypeptide comprising one or more fragments of ICP0, wherein the polypeptide comprises or consists of, from the N-terminus to the C-terminus: a first ICP0 sequence comprising a sequence having at least 80% sequence identity to SEQ ID NO: 1 and comprising no more than 30 amino acids: a first non-ICP0 sequence: a second ICP0 sequence comprising a sequence having at least 80% sequence identity to SEQ ID NO: 2 and comprising no more than 30 amino acids; and, optionally, a second non-ICP0 sequence; wherein the first and second non-ICP0 sequences have less than 10% sequence identity to ICP0, and wherein the mutant herpesvirus is substantially avirulent and immunogenic.

In some embodiments of the mutant herpesvirus comprising the mutant ICP0 polypeptide of the disclosure, the polypeptide lacks an ICP0 region selected from a RING finger region, an NLS region, and an oligomerization domain. In some embodiments of the mutant herpesvirus comprising the mutant ICP0 polypeptide of the disclosure, the polypeptide lacks two ICP0 regions selected from a RING finger region, an NLS region, and an oligomerization domain. In some embodiments of the mutant herpesvirus comprising the mutant ICP0 polypeptide of the disclosure, the polypeptide lacks a RING finger region, an NLS region, and an oligomerization domain.

In some embodiments of the mutant herpesvirus comprising the mutant ICP0 polypeptide of the disclosure, the first non-ICP0 sequence comprises at least 50, at least 100, at least 150, or at least 200 amino acids. In some embodiments of the mutant herpesvirus comprising the mutant ICP0 polypeptide of the disclosure, the first non-ICP0 sequence comprises at least 200 amino acids. In some embodiments of the mutant herpesvirus comprising the mutant ICP0 polypeptide of the disclosure, the first non-ICP0 sequence comprises 235 amino acids.

In some embodiments of the mutant herpesvirus comprising the mutant ICP0 polypeptide of the disclosure, the first non-ICP0 sequence is generated by a frameshift mutation or a nonsense mutation. In some embodiments of the mutant herpesvirus comprising the mutant ICP0 polypeptide of the disclosure, the first non-ICP0 sequence is predetermined and generated by a computer algorithm.

In some embodiments of the mutant herpesvirus comprising the mutant ICP0 polypeptide of the disclosure, the first non-ICP0 sequence comprises or consists of a florescent protein sequence. In some embodiments of the mutant herpesvirus comprising the mutant ICP0 polypeptide of the disclosure, the first non-ICP0 sequence comprises or consists of a green florescent protein (GFP) sequence. In some embodiments of the mutant herpesvirus comprising the mutant ICP0 polypeptide of the disclosure, the first non-ICP0 sequence has at least 80% sequence identity to SEQ ID NO: 3.

In some embodiments of the mutant herpesvirus comprising the mutant ICP0 polypeptide of the disclosure, the second non-ICP0 sequence comprises at least 50, at least 100, at least 150, or at least 200 amino acids. In some embodiments of the mutant herpesvirus comprising the mutant ICP0 polypeptide of the disclosure, the second non-ICP0 sequence comprises at least 100 amino acids. In some embodiments of the mutant herpesvirus comprising the mutant ICP0 polypeptide of the disclosure, the second non-ICP0 sequence comprises 124 amino acids.

In some embodiments of the mutant herpesvirus comprising the mutant ICP0 polypeptide of the disclosure, the second non-ICP0 sequence is generated by a frameshift mutation or a nonsense mutation. In some embodiments of the mutant herpesvirus comprising the mutant ICP0 polypeptide of the disclosure, the second non-ICP0 sequence is predetermined and generated by computer algorithm. In some embodiments of the mutant herpesvirus comprising the mutant ICP0 polypeptide of the disclosure, the second non-ICP0 sequence has at least 80% sequence identity to SEQ ID NO: 4. In some embodiments of the mutant herpesvirus comprising the mutant ICP0 polypeptide of the disclosure, the mutant ICP0) polypeptide lacks the second non-ICP0 sequence.

In some embodiments, the mutant herpesvirus comprises any of the mutant ICP0 polypeptides disclosed herein. In particular embodiments, the mutant herpesvirus is derived from HSV-1. In particular embodiments, the mutant herpesvirus is derived from HSV-2.

In some embodiments, the mutant herpesvirus, e.g., mutant HSV-2, comprises a mutant ICP0 polypeptide comprising or consisting of a polypeptide sequence having at least 80%, at least 85%, at least 90%, at least 95%, at least 98%, at least 99%, or 100% sequence identity to any one of SEQ ID NOs: 1-5 or 7-10. In certain embodiments, it comprises or consists of SEQ ID NO: 1 and SEQ ID NO: 2, or SEQ ID NO:5. In some embodiments of the mutant herpesvirus comprising the mutant ICP0 polypeptide of the disclosure, the polypeptide has at least 80%, at least 85%, at least 90%, at least 95%, at least 98%, at least 99%, or 100% sequence identity to SEQ ID NO: 5 or any other mutant ICP0 polypeptide disclosed herein.

In some embodiments, the disclosure provides a cell comprising a mutant herpesvirus and/or mutant ICP0 polypeptide disclosed herein. This can include cells that include genetic material that encodes for the production of the mutant herpesvirus and/or mutant ICP0 polypeptide. The cell can be any type of cell: however, it can be preferable for the cell to be a cell type that is capable of being infected with wild type HSV-1 and/or HSV-2. For example, the cell can be a host cell, such as a recombinant eukaryotic cell line containing the gene(s) encoding a mutant herpesvirus and/or mutant ICP0 polypeptide. In one embodiment, the cell can be a Vero cell, L7 cell, LLC-MK2 cell, or MDCK cell. Vero cells, an African green monkey kidney cell line, can be obtained from the American Type Cell Culture (ATCC, Manassas, Va.) and propagated in Dulbecco's modified Eagle's medium (DMEM) supplemented with 10% fetal bovine serum (FBS) as described previously in the art. Vero cells can be stably transformed with the gene (a) encoding for mutant HSV-1 and/or mutant ICP0, and passaged as described in the art.

Mutant ICP0 polypeptides and HSV comprising mutant ICP0 polypeptides may be produced using routine methods well known in the art. For example, mutant ICP0 polypeptides may be produced in a variety of cell lines using an expression vector comprising a promoter operably linked to a polynucleotide sequence encoding the mutant ICP0 polypeptide. Mutant virus may be produced, e.g., as described in U.S. Pat. No. 8,802,109.

### Immunogenic Composition

In some embodiments, the disclosure provides an immunogenic composition comprising a pharmaceutically acceptable carrier and the mutant herpesvirus of the disclosure. In some embodiments, the mutant herpesvirus, e.g., mutant HSV-2, comprises a mutant ICP0) polypeptide comprising or consisting of a polypeptide sequence having at least 80%, at least 85%, at least 90%, at least 95%, at least 98%, at least 99%, or 100% sequence identity to any one of SEQ ID NOs: 1-5 or 7-10. In certain embodiments, it comprises or consists of SEQ ID NO: 1 and SEQ ID NO: 2, or SEQ ID NO:5. The terms “immunogenic composition” and “vaccine” and “pharmaceutical composition” may be used interchangeably in the disclosure.

In some embodiments, the mutant herpesvirus used in the immunogenic composition of the disclosure may be derived from any alpha-herpesvirus that encode an ICP0-like protein. Methods of preparing vaccines to include a mutant virus are well established in the art of vaccines.

In certain embodiments, the disclosure provides a vaccine comprising the mutant herpesvirus of the disclosure, a formulation buffer, and, optionally, one or more cellular component from host cells that were used to propagate the live virus. In some embodiments, said cellular components comprise proteins, lipids, carbohydrates, and nucleic acids from said host cells. In particular embodiments, the nucleic acids from said host cells comprise fragments less than 200 nucleotides in length. In certain embodiments, the concentration of said nucleic acids is between 50 ng/ml and 3000 ng/ml. In some embodiments, the concentration of host cell protein is between 50 μg/mL and 500 μg/mL. In particular embodiments, the total protein concentration of said vaccine is between 400 μg/mL and 2000 μg/mL. The formulation buffer is typically any buffer suitable for administration to a subject. In certain embodiments, the formulation buffer maintains the pH of the vaccine composition within a physiologically acceptable range. In particular embodiments, said pH is between 6.0 and 8.0. In certain embodiments, said formulation buffer comprises phosphate buffered saline (PBS), L-glutamic acid monosodium salt monohydrate, and sucrose. Optionally, the L-glutamic acid monosodium salt monohydrate is present at about 0.25% and sucrose is present at about 1%. In certain embodiments, the osmolarity of the vaccine composition is in the range of 250-450 Osm/L.

In some embodiments, the mutant herpesvirus of the disclosure is present in the vaccine at a concentration higher than 1×10⁶ PFU/ml, higher than 2×10⁶ PFU/ml, higher than 3×10⁶ PFU/ml, higher than 4×10⁶ PFU/ml, higher than 5×10⁶ PFU/ml, higher than 1×10⁷ PFU/ml, or higher than 2×10⁷ PFU/ml.

In certain embodiments, the disclosure provides a unit dosage form of an immunogenic composition disclosed herein. In certain embodiments, the unit dosage form comprises between 1×10⁵ PFU and 1×10⁹ PFU of the mutant herpesvirus. In certain embodiments, the unit dosage form comprises between 1×10⁶ PFU and 1×10⁹ PFU of the mutant herpesvirus. In certain embodiments, the unit dosage form comprises between 1×10⁷ PFU and 1×10⁹ PFU of the mutant herpesvirus. In certain embodiments, the unit dosage form comprises between 1×10⁸ PFU and 1×10⁹ PFU of the mutant herpesvirus. In certain embodiments, the unit dosage form comprises about 1.0×10⁵ PFU, about 5×10⁵ PFU, about 1.0×10⁶ PFU, about 5×10⁶ PFU, about 1.0×10⁷ PFU, about 5×10⁷ PFU, about 1.0×10⁸ PFU, about 5×10⁸ PFU, or about 1×10⁹ PFU or the mutant herpesvirus. In certain embodiments, the unit dosage form comprises about 1.5×10⁸ PFU of the mutant herpesvirus. In certain embodiments, the unit dosage form comprises up to 1.5×10⁸ PFU of the mutant herpesvirus, and in certain embodiments, the unit dosage form comprises up to 2×10⁸ or up to 3×10⁸ PFU of the mutant herpesvirus. In certain embodiments, the unit dosage form is present in a container, e.g., a glass vial. The container may comprise one or more unit dosage form of the mutant herpesvirus.

In certain embodiments, the vaccine of the disclosure induces an immune response in a subject to whom the vaccine is administered. In certain embodiments, the immune response comprises an immune response to the mutant herpesvirus. In particular embodiments, the immune response comprises an immune response to a wild type herpesvirus. In certain embodiments, the vaccine induces an immune response against the mutant herpesvirus, wherein the induced immune response also recognizes wild type herpesvirus. In particular embodiments, the induced immune response provides a therapeutic and/or prophylactic effect against wild type herpesvirus.

Said immune response may comprise a cellular immune response, optionally mediated by Th1, Th2, and Th17. In some embodiments, said immune response comprises a humoral response, optionally comprising the induction of neutralizing antibodies. In some embodiments, said immune response comprises a cellular immune response, optionally mediated by Th1, Th2, and Th17, and a humoral response, optionally comprising the induction of neutralizing antibodies. In certain embodiments, the cellular immune response and/or humoral immune response is at least 10%, at least 20%, at least 30%, at least 40%, at least 50%, at least 60%, at least 70%, at least 80%, at least 90%, at least 2-fold, at least 3-fold, at least 4-fold, or at least five-fold greater than an immune response generated by another herpesvirus vaccine known in the art or the same herpesvirus but with the native ICP0 polypeptide.

In some embodiments, the mutant herpesvirus of the disclosure may be formulated into a vaccine using a stabilizer or other additive that includes native or recombinant serum albumin for this purpose. U.S. Pat. No. 6,210,683 provides representative conditions for this embodiment of the invention. U.S. Pat. Nos. 5,728,386, 6,051,238, 6,039,958 and 6,258,362 also contain details for stabilizers and methods for more gentle treatment of live virus vaccines. Stabilizers are often used in vaccine formulation, as vaccine potency may be adversely affected by concentration and storage conditions. Stabilizers often used for live vaccines of viruses such of measles, rubella and mumps generally include one or more saccharides, amino acids, sugar alcohols, gelatin and gelatin derivatives, to stabilize the virus and, in many cases keep the virus from denaturing during a concentration step.

In some embodiments, the mutant herpesvirus of the disclosure may be formulated into a vaccine or immunogenic composition comprising an adjuvant. “Adjuvant” as used in the disclosure refers to an agent that increases the immune response to an antigen (e.g., HSV-2 surface antigens). Examples of adjuvants include, but are not limited to, aluminum salts such as aluminum hydroxide gel (alum) or aluminum phosphate: Freund's Incomplete Adjuvant and Complete Adjuvant: Merck Adjuvant 65: an emulsion: a saponin, e.g. QS-21: a modified saponin: an unmethylated CpG dinucleotide: MF59; Montanide: ASO2: ASO4; ISCOM: a helper peptide: a TLR agonist: AS-2: MPL or 3d-MPL; LEIF; salts of calcium, iron or zinc: an insoluble suspension of acylated tyrosine: acylated sugars: cationically or anionically derivatized polysaccharides: polyphosphazenes: bio-degradable microspheres: monophosphoryl lipid A and quil A: Glucopyranosyl Lipid A (GLA): muramyl tripeptide phosphatidyl ethanolamine or an immunostimulating complex, including cytokines and immunostimulatory DNA sequences. In some embodiments, the adjuvant is QS-21, a saponin with a molecular formula of C₉₂O₄₆H₁₄₈. QS-21 is one of the most potent immunological adjuvants that has been widely used. Studies showed that QS-21 promoted high antigen-specific antibody responses and CD8⁺ T-cell response in mice and favored a balanced production of both IgG1 and IgG2a.

In some embodiments, the immunogenic compositions of the invention can be in the form of sterile injectable preparations, such as sterile injectable aqueous or oleaginous suspensions. For administration as injectable solutions or suspensions, the immunogenic compositions can be formulated according to techniques well-known in the art, using suitable dispersing or wetting and suspending agents, such as sterile oils, including synthetic mono- or diglycerides, and fatty acids, including oleic acid.

### Methods of Use

The disclosure provides methods for treatment and/or prevention of a viral infection, e.g., a herpesvirus (e.g., HSV-1 and/or HSV-2) infection in a subject, comprising administering to the subject a mutant virus comprising a mutant ICP0 as disclosed herein. In some embodiments, the mutant herpesvirus, e.g., mutant HSV-2, comprises a mutant ICP0) polypeptide comprising or consisting of a polypeptide sequence having at least 80%, at least 85%, at least 90%, at least 95%, at least 98%, at least 99%, or 100% sequence identity to any one of SEQ ID NOs: 1-5 or 7-10. In certain embodiments, it comprises or consists of SEQ ID NO: 1 and SEQ ID NO: 2, or SEQ ID NO:5. Methods of use include prophylactic methods and treatment methods. A subject may have been diagnosed as having a viral infection or considered at risk of being infected with said virus. Patients or subjects include mammals, such as human, and other primate, bovine, equine, canine, feline, porcine, and ovine animals.

The disclosure also provides methods for generating an immune response against a herpesvirus infection in a subject. The methods comprise administering to the subject an effective amount of a vaccine composition of the disclosure. The composition can be used as a therapeutic or prophylactic vaccine. The disclosure additionally provides methods of generating an immune response against a viral infection in the subject.

The disclosure provides a method of treating, suppressing, inhibiting, or preventing a herpesvirus infection in a subject, the method comprising administering to said subject an effective amount of any one of the aforementioned vaccines or immunogenic compositions. In some embodiments, the mutant herpesvirus, e.g., mutant HSV-2, comprises a mutant ICP0 polypeptide comprising or consisting of a polypeptide sequence having at least 80%, at least 85%, at least 90%, at least 95%, at least 98%, at least 99%, or 100% sequence identity to any one of SEQ ID NOs: 1-5 or 7-10. In certain embodiments, it comprises or consists of SEQ ID NO: 1 and SEQ ID NO: 2, or SEQ ID NO:5. In one embodiment, administration of said vaccine or immunogenic composition generates an immune response against the virus in the subject. In certain embodiments, said immune response comprises a cellular immune response, optionally mediated by Th1, Th2, and Th17. In particular embodiments, said immune response comprises a humoral response, optionally comprising the induction of neutralizing antibodies. In some embodiments, said immune response comprises a cellular immune response, optionally mediated by Th1, Th2, and Th17, and a humoral response, optionally comprising the induction of neutralizing antibodies. In one embodiment, said vaccine is administered intradermally, mucosally, intramuscularly, subcutaneously, sublingually, rectally, or vaginally.

The disclosure also provides a method of generating an immune response to a herpesvirus in a subject, the method comprising administering to the subject an effective amount of any one of the aforementioned vaccines. In some embodiments, the mutant herpesvirus, e.g., mutant HSV-2, comprises a mutant ICP0 polypeptide comprising or consisting of a polypeptide sequence having at least 80%, at least 85%, at least 90%, at least 95%, at least 98%, at least 99%, or 100% sequence identity to any one of SEQ ID NOs: 1-5 or 7-10. In certain embodiments, it comprises or consists of SEQ ID NO: 1 and SEQ ID NO: 2, or SEQ ID NO:5. In certain embodiments, said immune response comprises a cellular immune response, optionally mediated by Th1, Th2, and Th17. In particular embodiments, said immune response comprises a humoral response, optionally comprising the induction of neutralizing antibodies. In some embodiments, said immune response comprises a cellular immune response, optionally mediated by Th1, Th2, and Th17, and a humoral response, optionally comprising the induction of neutralizing antibodies. In one embodiment, said vaccine is administered intradermally, mucosally, intramuscularly, subcutaneously, sublingually, rectally, or vaginally.

The vaccine composition can be administered to a subject by any method known to a person skilled in the art, such as parenterally, transmucosally, transdermally, intramuscularly, intravenously, intra-dermally, intra-nasally, subcutaneously, intra-peritonealy, intra-ventricularly, intra-cranially, or intra-vaginally. In certain embodiments, vaccine compositions of the instant invention are administered via intradermal injection, epidermal injection, intramuscular injection, intradermal injection, subcutaneous injection, or intra-respiratory mucosal injection.

In certain embodiments, the vaccine compositions are administered by intravenous, intra-arterial, or intra-muscular injection of a liquid preparation. Suitable liquid formulations include solutions, suspensions, dispersions, emulsions, oils and the like. In another embodiment, the vaccine compositions are administered intravenously and are thus formulated in a form suitable for intravenous administration. In another embodiment, the vaccine compositions are administered intra-arterially and are thus formulated in a form suitable for intra-arterial administration. In another embodiment, the vaccine compositions are administered intramuscularly and are thus formulated in a form suitable for intra-muscular administration.

In one embodiment, the pharmaceutical compositions are administered by intradermal injection and are thus formulated in a form suitable for intradermal administration.

In some embodiments, the dose of the vaccine composition is administered to a patient in an amount sufficient to elicit an effective immune response to the specific antigens and/or to alleviate, reduce, cure or at least partially arrest symptoms and/or complications from the disease or infection. An amount adequate to accomplish this is defined as a “therapeutically effective dose.”

The dose can be determined by the activity of the composition produced and the condition of the patient, as well as the body weight or surface areas of the patient to be treated. The size of the dose can also be determined by the existence, nature, and extent of any adverse side effects that accompany the administration of a particular composition in a particular patient. In determining the effective amount of the composition to be administered in the treatment or prophylaxis of a viral infection, the physician may evaluate the production of an immune response against the virus, progression of the disease, and any treatment-related toxicity.

In some embodiments, the dose is between 1×10⁵ PFU and 1×10⁹ PFU of the mutant herpesvirus. In certain embodiments, the dose is about 1.0×10⁵ PFU, about 5×10⁵ PFU, about 1.0×10⁶ PFU, about 5×10⁶ PFU, about 1.0×10⁷ PFU, about 5×10⁷ PFU, about 1.0×10⁸ PFU, about 5×10⁸ PFU, or about 1×10⁹ PFU. In certain embodiments, the dose is about 1.5×10⁸ PFU. In certain embodiments, the unit dosage form comprises up to 1.5×10⁸ PFU of the mutant herpesvirus, and in certain embodiments, the unit dosage form comprises up to 2×10⁸ or up to 3×10⁸ PFU of the mutant herpesvirus.

In some embodiments, the vaccine includes an attenuated mutant herpesvirus of the disclosure that, after administration to the patient, can replicate to an amount that is within one log or 0.5 log of the amount of viral replication of the wild type or a reference virus. In some embodiments, the vaccine includes an attenuated herpesvirus of the disclosure that, after administration to the patient, replicates less than 50%, less than 10%, less than 1%, or less than 0.1% of the amount of viral replication of the wild type virus or reference virus.

Prophylaxis or treatment can be accomplished by administration at a single time point (single dose schedule) or multiple time points (multiple dose schedule). Administration can also be nearly simultaneous to multiple sites. In certain embodiments, the vaccine composition is administered to the subject as a single dose schedule. In certain embodiments, the vaccine composition is administered to the subject as a multiple dose schedule, e.g., a multiple dose schedule in which a primary course of vaccination with 1-6 separate doses elicits an immune response and is followed by other doses given at subsequent time intervals that maintain, reinforce, and/or boost the immune response to the virus. For example, in certain embodiments, a multiple dose schedule comprises initially providing the vaccine to a subject at time 0, and then providing a second dose to the subject 1-6 weeks after the initial dose, followed by providing to the subject a third dose 4-10 weeks after the initial dose, and if needed, providing one or more subsequent dose(s) after several weeks or several months.

Methods of the disclosure may be used to prevent an initial herpesvirus infection and to treat a herpesvirus infection, e.g., HSV-1 or HSV-2 infection, in a subject.

In some embodiments, the present invention provides a method of impeding, reducing, or inhibiting a viral infection, e.g., HSV-1 or HSV-2 infection, in a subject, the method comprising the step of administering to the subject an effective amount of a vaccine of the present invention.

In some embodiments, the mutant herpesvirus of the disclosure can be used in a vaccine or other composition to inhibit wild-type herpesvirus replication, and thereby inhibit or prevent the diseases caused by herpesvirus infections. In one embodiment, the mutant herpesvirus of the disclosure encoding the mutant ICP0 protein can be introduced into an individual that has been infected or may become infected with a herpesvirus virus. In some embodiments, the mutant herpesvirus of the disclosure can treat, limit, and/or prevent wild type herpesvirus infections as well as diseases caused by the wild type herpesvirus. For example, mutant HSV-1 viruses can be used to prevent wild-type HSV-1 infections and mutant HSV-2 viruses can be used to prevent wild-type HSV-2 infections. Likewise, any mutant alpha-herpesvirus of the disclosure encoding a mutant ICP0 protein can be used to prevent infection in the subject with its corresponding wild-type alpha-herpesvirus.

In some embodiments, the disclosure provides a method of treating a viral infection in a subject, the method comprising the step of administering to said subject an effective amount of a vaccine of the present invention. In one embodiment, the present invention provides a method of preventing or reducing the probability or likelihood of a subject becoming infected by a virus, the method comprising the step of administering to said subject an effective amount of a vaccine of the present invention. In some embodiments, the subject has not previously been infected with the virus. In some embodiments, the method reduces the probability or likelihood that the subject becomes infected upon contact with the virus or an infected mammal, e.g., human.

In some embodiments, the disclosure provides a method of treating a viral infection in a subject, the method comprising administering to the subject an effective amount of a vaccine composition of the present invention. In particular embodiments, the infection is an HSV-1 or HSV-2 infection, and the vaccine comprises the mutant HSV-1 or HSV-2 of the disclosure, respectively. In some embodiments, the treatment alleviates or improves one or more clinical symptoms or manifestations of the infection. For example, for HSV-1 or HSV-2, such symptoms include, but are not limited to local pain and/or burning sensation. In particular embodiments, the treatment reduces the impact of a herpes outbreak on a subject's daily life. Determining the effectiveness of the treatment may be determined, e.g., via patent-reported outcomes of symptoms, and patients may track symptoms and/or the effect of a herpes outbreak on daily life in a journal or diary. The method may be used to treat mild or moderate to severe episodes. Mild episodes are characterized by no vesicles or appearance of singular vesicles without pain or discomfort interfering with usual daily activities. Moderate to severe episodes or outbreaks present as cluster of vesicles that are associated with pain and discomfort that interferes with usual daily activities. In particular embodiments, HSV-1 or HSV-2 vaccine compositions prepared as disclosed are used to treat moderate to severe episodes of HSV-1 or HSV-2 infection, respectively.

In some embodiments, the disclosure provides a method of preventing or inhibiting a recurrence following a primary viral infection in a subject, the method comprising the step of administering to said subject an effective amount of a vaccine of the disclosure. In one embodiment, the virus is HSV-2 and a “recurrence” (also sometimes called outbreaks, episodes, or flare-ups) are repeat symptoms (e.g., sores, blisters, patches of red skin or tiny splits) which appear at or close to the place where the infection was first noticed. A recurrence may comprise reinfection of skin tissue following latent neuronal HSV-2 infection, reactivation of HSV-2 after a latency period, or symptomatic HSV-2 lesions following a non-symptomatic latency period. In some embodiments, the recurrence is a recurrence of herpes, e.g., oral or genital herpes. In some embodiments, the method reduces the number of recurrences that occur within one year, two years, or five years, e.g., by at least 10%, at least 20%, at least 30%, at least 40%, at least 50%, at least 60%, at least 70%, at least 80%, at least 90% or 100%. In some embodiments, the method reduces the frequency of recurrences that occur over the course of one year, two years, or five years. In certain embodiments, the frequency of recurrence over the time period is reduced by at least 10%, at least 20%, at least 30%, at least 40%, at least 50%, at least 60%, at least 70%, at least 80%, at least 90% or 100%. In certain embodiments, the frequency of recurrence is reduced to less than once per two months, less than once per four months, less than once per six months, less than once per year, less than once per two years, or less than once per five years.

In the case of HSV, after the primary infection, HSV becomes latent in the cell bodies of nerves in the area. Some infected people experience sporadic episodes of viral reactivation, followed by transportation of the virus via the nerve's axon to the skin, where virus replication and shedding occurs. Herpes is contagious if the carrier is producing and shedding the virus. In some embodiments, the disclosure provides a method of inhibiting HSV-2 replication or HSV-2 shedding by an infected subject, the method comprising the step of administering to said subject an effective amount of a vaccine of the present invention. In certain embodiments, the level of shedding is reduced by at least 10%, at least 20%, at least 30%, at least 40%, at least 50%, at least 60%, at least 70%, at least 80%, at least 90% or 100%.

In some embodiments, the disclosure provides a method of preventing or reducing the severity or duration of HSV-2 labialis following a primary HSV-2 infection in a subject, the method comprising the step of administering to said subject an effective amount of a vaccine of the disclosure. In some embodiments, the duration is reduced to less than four weeks, less than three weeks, less than two weeks, less than one week, less than 3 days, or less than 1 day. Sometimes, an infection by an HSV-2 is marked by watery blisters in the skin or mucous membranes of the mouth, lips, or genitals. Lesions heal with a scab characteristic of herpetic disease. The severity of any of these symptoms may be reduced. However, the infection is persistent, and symptoms may recur periodically as outbreaks of sores near the site of original infection.

In one embodiment, the disclosure provides a method of treating, suppressing or inhibiting an HSV genital infection, e.g. genital herpes, the method comprising the step of administering to said subject an effective amount of a vaccine of the disclosure. In one embodiment, the disclosure provides a method of treating, suppressing or inhibiting an HSV oral infection, e.g. oral herpes, the method comprising the step of administering to said subject an effective amount of a vaccine of the disclosure.

In one embodiment, the disclosure provides a method of diminishing the severity of a recurrence of a viral infection, the method comprising the step of administering to said subject an effective amount of a vaccine of the disclosure. In one embodiment, the disclosure provides a method of reducing the frequency of a recurrence of a viral infection, the method comprising the step of administering to said subject an effective amount of a vaccine of the disclosure. In one embodiment, the disclosure provides any of the described methods in an infected subject.

In one embodiment, the disclosure provides a method of vaccinating a subject against a herpesvirus infection, the method comprising the step of administering to said subject an effective amount of a vaccine of the disclosure. In one embodiment, the disclosure provides a method of suppressing a herpesvirus infection in a subject, the method comprising the step of administering to said subject an effective amount of a vaccine of the disclosure. In one embodiment, the present invention provides a method of impeding a viral infection in a subject, the method comprising the step of administering to said subject an effective amount of a vaccine of the disclosure.

It is to be understood that the methods of the disclosure may be used to treat, inhibit, or suppress a herpesvirus infection or primary or secondary symptoms related to such an infection following exposure of the subject to said herpesvirus. In one embodiment, the subject has been infected with the virus before vaccination. In another embodiment, the subject is at risk for viral infection. In another embodiment, whether or not the subject has been infected with the virus at the time of vaccination, vaccination by a method of the disclosure is efficacious in treating, inhibiting, suppressing the viral infection or primary or secondary symptoms related to such an infection.

In some embodiments of any of the methods disclosed herein, administration of the vaccine composition to a subject diagnosed with a herpesvirus infection or at risk of being infected with said virus will elicit an immune response in the subject. In certain embodiments, immune responses include both cell-mediated immune responses (responses mediated by antigen-specific T cells and non-specific cells of the immune system-Th1, Th2, Th17) and humoral immune responses (responses mediated by antibodies).

In certain embodiments, a mammal, e.g., a human, is immunized with a vaccine of the disclosure and then boosted one or more times with the vaccine. In one embodiment, the mammal is boosted about 2 to about 4 weeks after the initial administration of the vaccine. If the mammal is to be boosted more than once, there may be about a 2 to 12-week interval between boosts. In one embodiment, the mammal is boosted at about 12 weeks and about 36 weeks after the initial administration of the vaccine. The dose used to boost the immune response may include one more cytokines, chemokines, or immunomodulators not present in the priming dose of the vaccine.

### Additional Definitions

Unless otherwise defined in the disclosure, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described in the disclosure, are those well-known and commonly used in the art.

As used in the disclosure, the following terms have the meanings ascribed to them unless specified otherwise.

The articles “a.” “an.” and “the” are used in the disclosure to refer to one or to more than one (i.e. to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used in the disclosure, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise”, “comprises,” and “comprising” will be understood to imply the inclusion of a stated step or element or group of steps or elements but not the exclusion of any other step or element or group of steps or elements. In some embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

A “subject,” “individual,” or “patient” as used in the disclosure, includes any animal that exhibits a symptom of a condition that can be detected or identified with compositions of the disclosure. Suitable subjects include laboratory animals (such as mouse, rat, rabbit, or guinea pig), farm animals (such as horses, cows, sheep, pigs), and domestic animals or pets (such as a cat or dog). In some embodiments, the subject is a mammal. In certain embodiments, the subject is a non-human primate and, in some embodiments, the subject is a human.

The terms “administering” or “introducing” or “providing”, as used herein, refer to delivery of a composition to a cell, to cells, tissues and/or organs of a subject, or to a subject. Such administering or introducing may take place in vivo, in vitro or ex vivo.

The terms “treatment”, “treating” and the like are used herein to generally mean obtaining a desired pharmacologic and/or physiologic effect. The effect may be prophylactic in terms of completely or partially preventing a disease, infection, or symptom thereof, e.g. reducing the likelihood that the disease, infection, or symptom thereof occurs in the subject, and/or may be therapeutic in terms of a partial or complete cure for a disease, infection, and/or adverse effect attributable to the disease or infection “Treatment” as used herein covers any treatment of a disease or disorder (e.g., an infection, such as a herpesvirus infection) in a mammal, and includes: (a) preventing the disease or disorder from occurring in a subject which may be predisposed to the disease but has not yet been diagnosed as having it: (b) inhibiting the disease or disorder, i.e., arresting its development: or (c) relieving the disease or disorder, i.e., causing regression of the disease or disorder. The therapeutic agent may be administered before, during or after the onset of disease or disorder. The treatment of ongoing disease or disorder, where the treatment stabilizes or reduces the undesirable clinical symptoms of the patient, is of particular interest. The subject therapy may be administered during the symptomatic stage of the disease or disorder, and in some cases after the symptomatic stage of the disease.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the worldwide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. A detailed description of the FASTA algorithm is available over the worldwide web at https://bip.weizmann.ac.il/education/materials/gcg/fasta.html. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00: Gap Size Penalty: 0.33: and Joining Penalty: 30.0.

The term “native” or “wild-type” as used herein refers to a nucleotide sequence, e.g., gene, or gene product, e.g. RNA or protein, that is present in a wild-type cell, tissue, organ or organism. The term “variant” or “mutant” as used herein encompasses a mutant of a reference polynucleotide or polypeptide sequence, for example a native polynucleotide or polypeptide sequence, i.e., having less than 100% sequence identity with the reference polynucleotide or polypeptide sequence. Put another way, a variant or mutant comprises at least one amino acid difference (e.g., amino acid substitution, amino acid insertion, amino acid deletion) relative to a reference polynucleotide sequence, e.g., a native polynucleotide or polypeptide sequence. For example, a variant or mutant may be a polynucleotide having a sequence identity of 50% or more, 60% or more, or 70% or more with a full-length native polynucleotide sequence, e.g., an identity of 75% or 80% or more, such as 85%, 90%, or 95% or more, for example, 98% or 99% identity with the full-length native polynucleotide sequence. As another example, a variant or mutant may be a polypeptide having a sequence identity of 70% or more with a full-length native polypeptide sequence, e.g., an identity of 75% or 80% or more, such as 85%, 90%, or 95% or more, for example, 98% or 99% identity with the full-length native polypeptide sequence. Variants may also include variant fragments of a reference, e.g., native, sequence sharing a sequence identity of 70% or more with a fragment of the reference, e.g., native, sequence, e.g., an identity of 75% or 80% or more, such as 85%, 90%, or 95% or more, for example, 98% or 99% identity with the native sequence. Variants also include variant cells, tissues, organs or organisms comprising the variant polynucleotide or polypeptide.

Reference to “increase” or “enhance” and the like can mean an increase of any amount as compared to a reference value, for example, an increase of at least 10%, at least 20%, at least 30%, at least 40%, at least 50%, at least 60%, at least 70%, at least 80%, at least 90%, at least two-fold, at least five-fold, at least 10-fold, or at least 20-fold. Reference to “inhibit” or “decrease” or the like can mean a decrease of any amount as compared to a reference value, for example, a decrease of at least 10%, at least 20%, at least 30%, at least 40%, at least 50%, at least 60%, at least 70%, at least 80%, at least 90%. In certain embodiments, a reference value may be a predetermined value, and in certain embodiments a reference value may be a control value or other determined value. In particular embodiments, a reference value may be the value determined without a treatment disclosed herein, wherein the treatment results in a comparative increase or decrease of the characteristic being measured or described.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

Furthermore, it is intended that any method described in the disclosure may be rewritten into Swiss-type format for the use of any agent described in the disclosure, for the manufacture of a medicament, in treating any of the disorders described in the disclosure. Likewise, it is intended for any method described in the disclosure to be rewritten as a compound for use claim, or as a use of a compound claim.

All publications, patents, and patent applications described in the disclosure are hereby incorporated by reference in their entireties.

### EXAMPLES

The disclosure is further illustrated by the following examples, which are not to be construed as limiting this disclosure in scope or spirit to the specific procedures described in the disclosure. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure.

### Example 1

### Mutant HSV-2 ICP0 Protein

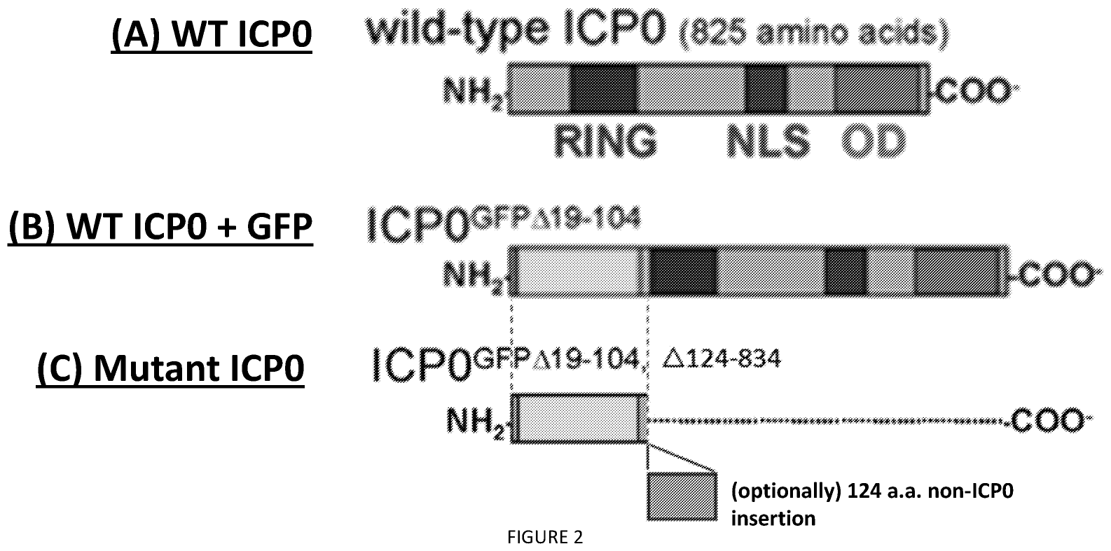

A mutant HSV-2 strain MS having a mutant ICP0 protein was generated, according to methods generally described in U.S. Pat. No. 8,802,109, and the genome of the mutant HSV-2 was sequenced by next-generation sequencing (NGS). The sequence of the mutated ICP0 gene was confirmed by Sanger sequencing, and a schematic illustration of the mutated ICP0 is depicted in FIG. 2(C). The mutant ICP0 comprises, from the N terminus to the C terminus, a first 19 amino acid (a.a.) sequence of the wild type ICP0 (SEQ ID NO: 1), a GFP protein sequence insertion (SEQ ID NO: 3), a second 19 a.a sequence of the wild type ICP0 (SEQ ID NO: 2), and a 124 a.a. non-ICP0 sequence (SEQ ID NO: 4). The full length mutant ICP0 comprises SEQ ID NO: 5 and is encoded by a polynucleotide comprising SEQ ID NO: 6. Table 1 lists the sequences of the mutant ICP0 in this example.

| SEQ | | |
|---|---|---|
| ID NO | Description | Amino acid (a.a.) or DNA Sequence |
| 1 | First ICP0 | MEPRPGTSSRADPGPERP |
| | sequence (a.a.) | |
| 2 | Second ICP0 | GSCGGGPVGEEEAEAGGGG |
| | sequence (a.a.) | |
| 3 | First non-ICP0 | LSKGEELFTGVVPILVELDGDVNGHKFSVSGEGEGDATYG |
| | sequence (a.a.) | KLTLKFICTTGKLPVPWPTLVTTLTYGVQCFSRYPDHMKQ |
| | | HDFFKSAMPEGYVQERTIFFKDDGNYKTRAEVKFEGDTLV |
| | | NRIELKGIDFKEDGNILGHKLEYNYNSHNVYIMADKQKNGI |
| | | KVNFKIRHNIEDGSVQLADHYQQNTPIGDGPVLLPDNHYLS |
| | | TQSALSKDPNEKRDHMVLLEFVTAAGITLGMDEL |
| 4 | Second non- | RRVRRVHGRDRPAPALPEFSLPAPLLHPVHEDLDSVAQHVS |
| | ICP0 sequence | PVQHPGGVPDSGRDRQRVVQHHPDSERPPDPRGGRGGRA |
| | (a.a.) | VRHGRGLYLDGQPADGPALPVAGGTHGPRPVAHPPVARH |
| | | GRRGR |
| 5 | Full-length | MEPRPGTSSRADPGPERPLSKGEELFTGVVPILVELDGDVN |
| | mutant | GHKFSVSGEGEGDATYGKLTLKFICTTGKLPVPWPTLVTTL |
| | sequence (a.a.) | TYGVQCFSRYPDHMKQHDFFKSAMPEGYVQERTIFFKDDG |
| | | NYKTRAEVKFEGDTLVNRIELKGIDFKEDGNILGHKLEYNY |
| | | NSHNVYIMADKQKNGIKVNFKIRHNIEDGSVQLADHYQQN |
| | | TPIGDGPVLLPDNHYLSTQSALSKDPNEKRDHMVLLEFVTA |
| | | AGITLGMDELGSCGGGPVGEEEAEAGGGGRRVRRVHGRD |
| | | RPAPALPEFSLPAPLLHPVHEDLDSVAQHVSPVQHPGGVPD |
| | | SGRDRQRVVQHHPDSERPPDPRGGRGGRAVRHGRGLYLD |
| | | GQPADGPALPVAGGTHGPRPVAHPPVARHGRRGR |
| 6 | Full-length | ATGGAACCCCGGCCCGGCACGAGCTCCCGGGCGGACCC |
| | mutant | CGGCCCCGAGCGGCCGCTGAGCAAGGGCGAGGAGCTGT |
| | sequence | TCACCGGGGTGGTGCCCATCCTGGTCGAGCTGGACGGCG |
| | (DNA) | ACGTAAACGGCCACAAGTTCAGCGTGTCCGGCGAGGGC |
| | | GAGGGCGATGCCACCTACGGCAAGCTGACCCTGAAGTTC |
| | | ATCTGCACCACCGGCAAGCTGCCCGTGCCCTGGCCCACC |
| | | CTCGTGACCACCCTGACCTACGGCGTGCAGTGCTTCAGC |
| | | CGCTACCCCGACCACATGAAGCAGCACGACTTCTTCAAG |
| | | TCCGCCATGCCCGAAGGCTACGTCCAGGAGCGCACCATC |
| | | TTCTTCAAGGACGACGGCAACTACAAGACCCGCGCCGA |
| | | GGTGAAGTTCGAGGGCGACACCCTGGTGAACCGCATCG |
| | | AGCTGAAGGGCATCGACTTCAAGGAGGACGGCAACATC |
| | | CTGGGGCACAAGCTGGAGTACAACTACAACAGCCACAA |
| | | CGTCTATATCATGGCCGACAAGCAGAAGAACGGCATCA |
| | | AGGTGAACTTCAAGATCCGCCACAACATCGAGGACGGC |
| | | AGCGTGCAGCTCGCCGACCACTACCAGCAGAACACCCCC |
| | | ATCGGCGACGGCCCCGTGCTGCTGCCCGACAACCACTAC |
| | | CTGAGCACCCAGTCCGCCCTGAGCAAAGACCCCAACGA |
| | | GAAGCGCGATCACATGGTCCTGCTGGAGTTCGTGACCGC |
| | | CGCCGGGATCACTCTCGGCATGGACGAGCTGGGATCCTG |
| | | CGGGGGTGGGCCCGTGGGTGAGGAGGAAGCGGAAGCGG |
| | | GAGGGGGGGGGCGACGTGTGCGCCGTGTGCACGGACGA |
| | | GATCGCCCCGCCCCTGCGCTGCCAGAGTTTTCCCTGCCT |
| | | GCACCCCTTCTGCATCCCGTGCATGAAGACCTGGATTCC |
| | | GTTGCGCAACACGTGTCCCCTGTGCAACACCCCGGTGGC |
| | | GTACCTGATAGTGGGCGTGACCGCCAGCGGGTCGTTCAG |
| | | CACCATCCCGATAGTGAACGACCCCCGGACCCGCGTGGA |
| | | GGCCGAGGCGGCCGTGCGGTCCGGCACGGCCGTGGACT |
| | | TTATCTGGACGGGCAACCCGCGGACGGCCCCGCGCTCCC |
| | | TGTCGCTGGGGGGACACACGGTCCGCGCCCTGTCGCCCA |
| | | CCCCCCCGTGGCCCGGCACGGACGACGAGGACGATGA |

### Additional Sequences:

SEQ ID NO: 7
MEPRPGTSSRADPGPERPGSCGGGPVGEEEAEAGGGG
SEQ ID NO: 8
MEPRPGTSSRADPGPERPGSCGGGPVGEEEAEAGGGGRRVRRVHGRDRP
APALPEFSLPAPLLHPVHEDLDSVAQHVSPVQHPGGVPDSGRDRQRVVQ
HHPDSERPPDPRGGRGGRAVRHGRGLYLDGQPADGPALPVAGGTHGPRP
VAHPPVARHGRRGR
SEQ ID NO: 9
MEPRPGTSSRADPGPERPLSKGEELFTGVVPILVELDGDVNGHKFSVSG
EGEGDATYGKLTLKFICTTGKLPVPWPTLVTTLTYGVQCFSRYPDHMKQ
HDFFKSAMPEGYVQERTIFFKDDGNYKTRAEVKFEGDTLVNRIELKGID
FKEDGNILGHKLEYNYNSHNVYIMADKQKNGIKVNFKIRHNIEDGSVQL
ADHYQQNTPIGDGPVLLPDNHYLSTQSALSKDPNEKRDHMVLLEFVTAA
GITLGMDELGSCGGGPVGEEEAEAGGGG
SEQ ID NO: 10
MEPRPGTSSRADPGPERPLSKGEELFTGVVPILVELDGDVNGHKFSVSG
EGEGDATYGKLTLKFICTTGKLPVPWPTLVTTLTYGVQCFSRYPDHMKQ
HDFFKSAMPEGYVQERTIFFKDDGNYKTRAEVKFEGDTLVNRIELKGID
FKEDGNILGHKLEYNYNSHNVYIMADKQKNGIKVNFKIRHNIEDGSVQL
ADHYQQNTPIGDGPVLLPDNHYLSTQSALSKDPNEKRDHMVLLEFVTAA
GITLGMDELGSCGGGPVGEEEAEAGGGGRRVRRVHGRDRPAPALPEFSL
PAPLLHPVHEDLDSVAQHVSPVQHPGGVPDSGRDRQRVVQHHPDSERPP
DPRGGRGGRAVRHGRGLYLDGQPADGPALPVAGGTHGPRPVAHPPVARH
GRRGR

### Example 2

### Toxicity Study

The purpose of this study was to assess the systemic toxic potential and biodistribution of an HSV-2 vaccine comprising the mutant ICP0 sequence described in Example 1 (SEQ ID NO:5: (an HSV-2 genetically modified, live attenuated viral vaccine) via four intradermal administrations (administered once every two weeks on Days 1, 15, 29 and 43) in CD-1 mice, followed by a 3-week post treatment observation period.

Two groups, each comprising 12 male and 12 female CD-1 [Crl:CD1(ICR)] mice, received this HSV-2 vaccine at a fixed dose (low dose: 4 times 1.23×10⁶ PFU/5 μl/animal: high dose: four times 1.23×10⁷ PFU/50 μl/animal). A similarly constituted control group received the vehicle, physiological saline, at the same volume dose as the high dose treated group (50 μl/injection). A further six male and six female CD-1 mice were assigned to each of the control and high dose groups. These animals were treated for six weeks, followed by a three-week period without treatment to assess the full formation of the immune response and the potential for any treatment-related change to recover. A further five males and five females were allocated to the control group and treated groups for qPCR evaluation.

During the study, clinical condition, body weight, food consumption, body temperature, ophthalmic examination, hematology (peripheral blood), blood chemistry, immunogenicity, qPCR analysis, organ weight, macropathology, and histopathology investigations were undertaken. Group mean weight changes and standard deviations were calculated from the weight changes of individual animals. Group mean food consumptions and standard deviations for each period were derived from unrounded cage values. Overall mean food consumption values were calculated for each cage and the mean of these cage means were calculated for each group/sex. Group mean body temperatures and standard deviations for each period were derived from individual values. Overall mean values were calculated for each group/sex. Blood samples (nominally 0.3 mL) were withdrawn from the orbital sinus, collected into tubes containing EDTA anticoagulant and examined for the following characteristics using a Bayer Advia 120 analyzer:
Hematocrit (Hct)
Hemoglobin concentration (Hb)
Erythrocyte count (RBC)
Absolute reticulocyte count (Retic)
Mean cell hemoglobin (MCH)
Mean cell hemoglobin concentration (MCHC)
Mean cell volume (MCV)
Red cell distribution width (RDW)
Total leucocyte count (WBC)
Differential leucocyte count:
Neutrophils (N)
Lymphocytes (L)
Eosinophils (E)
Basophils (B)
Monocytes (M)
Large unstained cells (LUC)
Platelet count (Plt).

### Results

The vaccine was well tolerated with no systemic sign of toxicity. There was no death nor effect of treatment on bodyweight gain, body temperature, food consumption and there was no ophthalmic finding. There was no pyrogenic effect following administration.

Haematological analysis of the peripheral blood three days after the fourth administration revealed higher than control mean total leucocyte numbers in males that received 1.23×10⁷ PFU RVx201/animal; this was associated with higher than control mean neutrophil, lymphocyte, eosinophil and monocyte numbers. Three weeks after the fourth administration, higher than control mean lymphocyte numbers were apparent in males that received four times 1.23×10⁷ PFU RVx201 administrations.

Biochemical evaluation of the plasma three days and three weeks after the fourth administration revealed no treatment related effect.

Anti HSV-2 vaccine IgG antibody responses were detectable in most animals from the third low dose of the vaccine administered (1.23×10⁶ PFU vaccine/animal) and from the second dose of the high dose administered (1.23×10⁷ PFU vaccine/animal). The animals treated with the high dose maintained this positive response after the three-week extended observation period.

The HSV-2 vaccine was found with quantifiable results in five skin samples from injection sites in both dose groups with slightly higher DNA copy numbers in the high dose group (1.23×10⁷ PFU vaccine/animal). All injection sites obtained from the control animals were negative for the HSV-2 vaccine. Most injection sites from the high dose group (1.23×10⁷ PFU vaccine/animal) in the were negative for the HSV-2 vaccine at the end of the extended observation phase. All other tissues analysed were free of the HSV-2 vaccine at the end of the treatment period. The HSV-2 vaccine was not found in serum samples, blood clots and was not shed to the environment.

Higher than control absolute and body weight adjusted spleen weights were observed in males and females administered both the low and the high dose, the variations were no longer observed at the end of the extended observation phase.

There was no test item-related macroscopic finding. Histopathology changes were evident two days after the last administration in the spleen (dose related increased extramedullary haemopoiesis and germinal centres) and injection sites (inflammatory cell infiltrate) of males and females administered both, low and high dose of RVx201 and had exhibited full recovery at the end of the extended observation phase.

### Conclusion

These studies demonstrate that four intradermal injections of the HSV-2 vaccine to CD-1 mice, each given at two-week intervals, was without sign of toxicity. Haematological and histopathological signs indicative of immune stimulation were evident.

### Example 3

### Prophylactic Efficacy

This study demonstrates the efficacy of the herpes vaccine of Examples 1 and 2 via immunization followed by vaginal challenge with HSV-2 in CD-1 mice. This study analyzed immune response in the live host to immunization followed by a challenge with wild type pathogen.

A total of Forty (40) female CD-1 mice of six (6) weeks of age were used in the study. Mice were divided into five (5) groups of eight (8) animals each: groups 1 & 2 were control groups immunized with saline, while groups 3, 4 & 5 were immunized with low (1×10⁵ PFU/animal), medium (1×10⁶ PFU/animal) or high (1×10⁷ PFU/animal) doses of the HSV-2 vaccine, respectively. The dosing groups and study schedule are shown in Table 1.

TABLE 1
Vaccine Dosing Groups

| | | Group | | Blood | Body | Vaginal | Clinical | |
|---|---|---|---|---|---|---|---|---|
| Group | N | Designation | Dosing | Collection | Weights | Swabs | Observations | Necropsy |
| 1 | 8 | Control | SC: -Day 0-left | -Day 0 and | Day −1, 2, | Daily from | Daily | Day |
| | | (Saline) | footpad; Day | 60 -pre- | 7, 14, 21, | Day 91 to | | 120 |
| | | | 30-right | dose Day | 28, 35, 42, | 97 (1 | | |
| | | | footpad | 90 pre- | 49, 56, 63, | through 7 | | |
| | | | | challenge. | 70, 77, 84, | days post- | | |
| | | | | Day 120 | 90, 92, 96, | challenge) | | |
| | | | | terminal | 103, 110, | | | |
| | | | | | 117 | | | |
| 2 | 8 | Control | SC: -Day 0-left | -Day 0 and | Day −1, 2, | Daily from | Daily | Day |
| | | (Saline) + | footpad; Day | 60 -pre- | 7, 14, 21, | Day 91 to | | 120 |
| | | infection | 30-right | dose Day | 28, 35, 42, | 97 (1 | | |
| | | | footpad | 90 pre- | 49, 56, 63, | through 7 | | |
| | | Vaginal | SC -Day 83 & | challenge. | 70, 77, 84, | days post- | | |
| | | Challenge | Day 87- pre- | Day 120 | 90, 92, 96, | challenge) | | |
| | | | infection | terminal | 103, 110, | | | |
| | | | Intravaginal: | | 117 | | | |
| | | | Day 90- HSV- | | | | | |
| | | | 2 challenge | | | | | |
| 3 | 8 | Low Dose | SC: -Day 0-left | -Day 0 and | Day −1, 2, | Daily from | Daily | Day |
| | | (1 × 10⁵ | footpad; Day | 60 -pre- | 7, 14, 21, | Day 91 to | | 120 |
| | | PFU/animal) | 30-right | dose Day | 28, 35, 42, | 97 (1 | | |
| | | | footpad | 90 pre- | 49, 56, 63, | through 7 | | |
| | | Vaginal | SC -Day 83 & | challenge. | 70, 77, 84, | days post- | | |
| | | Challenge | Day 87- pre- | Day 120 | 90, 92, 96, | challenge) | | |
| | | | infection | terminal | 103, 110, | | | |
| | | | Intravaginal: | | 117 | | | |
| | | | Day 90- HSV- | | | | | |
| | | | 2 challenge | | | | | |
| 4 | 8 | Mid-dose | SC: -Day 0-left | -Day 0 and | Day −1, 2, | Daily from | Daily | Day |
| | | (1 × 10⁶ | footpad; Day | 60 -pre- | 7, 14, 21, | Day 91 to | | 120 |
| | | PFU/animal) | 30-right | dose Day | 28, 35, 42, | 97 (1 | | |
| | | | footpad | 90 pre- | 49, 56, 63, | through 7 | | |
| | | Vaginal | SC -Day 83 & | challenge. | 70, 77, 84, | days post- | | |
| | | Challenge | Day 87- pre- | Day 120 | 90, 92, 96, | challenge) | | |
| | | | infection | terminal | 103, 110, | | | |
| | | | Intravaginal: | | 117 | | | |
| | | | Day 90- HSV- | | | | | |
| | | | 2 challenge | | | | | |
| 5 | 8 | High Dose | SC: -Day 0-left | -Day 0 and | Day −1, 2, | Daily from | Daily | Day |
| | | (1 × 10⁷ | footpad; Day | 60 -pre- | 7, 14, 21, | Day 91 to | | 120 |
| | | PFU/animal) | 30-right | dose Day | 28, 35, 42, | 97 (1 | | |
| | | | footpad | 90 pre- | 49, 56, 63, | through 7 | | |
| | | Vaginal | SC -Day 83 & | challenge. | 70, 77, 84, | days post- | | |
| | | Challenge | Day 87- pre- | Day 120 | 90, 92, 96, | challenge) | | |
| | | | infection | terminal | 103, 110, | | | |
| | | | Intravaginal: Day | | 117 | | | |
| | | | 90- HSV-2 | | | | | |
| | | | challenge | | | | | |

Immunization was performed via footpad injection, with two treatments at an interval of thirty (30) days (day 0 and day 30). The first treatment was administered into the left footpad, and the second treatment was administered into the right footpad. On Days 83 and 87, groups 2, 3, 4, and 5 received a pre-treatment via the subcutaneous route with 2-mg/animal medroxyprogesterone (Medroxyprogesterone Acetate, Amphastar Pharmaceuticals, [Kaushic et al., 2003]). On Day 90, groups 2, 3, 4, and 5 were challenged intravaginal with wild type HSV-2 MS (5×10⁵ PFU) under BLS-2 conditions.

Clinical observations were conducted daily, and body weights were measured before administration, two (2) days after administration, and then once a week until study termination. Blood was collected for serum processing at five (5) timepoints-pre-immunization (i.e., Day 0), pre-boost, Day 60, pre-challenge (Day 90), and end of in-life (Day 120). Serum from groups 1 & 3-5 were assessed via a neutralization assay, which measures the ability of the antibodies in the serum to protect cells against infection from live virus in vitro by using a serum dilution series to determine the concentration of serum (the neutralizing antibody titer) that reduces the number of plaques by 50% as compared to the serum-free virus. Vaginal swabs were collected from all animals on Days 1-7 post-challenge (Study Days 91 to 97). On Day 120, animals underwent necropsy with a special emphasis on macroscopic evaluation to target tissues of HSV-2 infection. The following tissues were collected for histopathology: all macroscopic findings, injection site, dorsal root ganglion (DRG) on the levels of the injection site, sacral sympathetic ganglia (SSG), spinal cord, skin lesions, lymph nodes, spleen, kidney, liver, heart, lung, spinal cord, and brain.

Before viral challenge, mice were observed every other day except weekends for visible signs of morbidity, hunched posture, ruffled fur, lethargy, diarrhea, and/or loss of >20% body weight. Following viral challenge, clinical observations for HSV-2-related disease severity were conducted daily using a scoring matrix with criteria of 0 to 3 (0=normal, 1=mild, 2=moderate, 3=severe) for each of three individual phenotypes of fur loss, urinary retention, and hind limb weakness or paralysis (0=normal, 1=mild weakness, 2=mild paralysis, 3=severe paralysis); and a scoring of 0 to 4: (0=no disease, 1=erythema, 2=single or few small lesions, 3=3-5 lesions or coalescence of lesions, 4=6+ lesions or coalescence of lesions, ulcerated lesions) for severity.

### Results

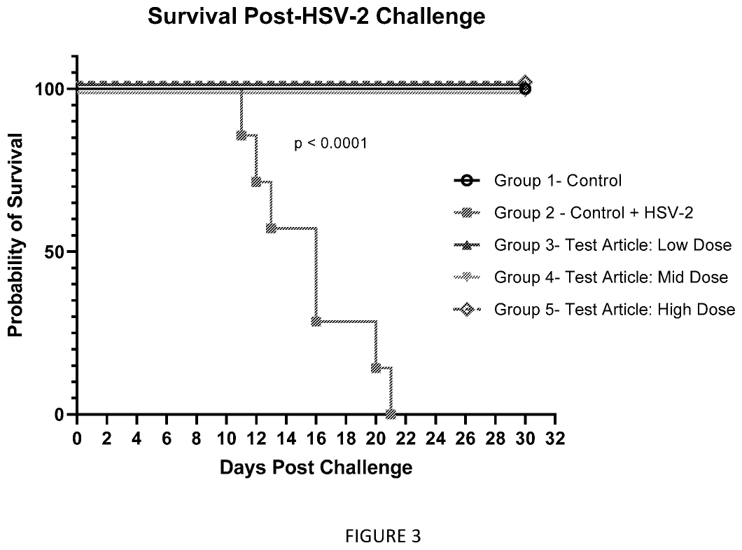

One animal of Group 2 was found missing before viral challenge. Seven animals assigned to Group 2 were found dead following viral challenge on Day 101, Day 102, Day 103: Day 106; and Day 110. Survival curves for animals post-HSV-2 challenge are presented in FIG. 3. All animals in Groups 1, 3, 4 and 5 survived the in-life portion of the study for 120 days. The median survival for Group 2 animals following viral challenge was 16 days.

All animals in all Groups 1 to 5 were scored as healthy prior to viral challenge on Day 90. Following viral challenge, clinical signs of HSV-2-related disease were evaluated daily using a severity scoring matrix. Onset of clinical signs of HSV-2-related disease including fur loss, urinary retention, and hindlimb weakness/paralysis was first observed on Day 10 post-challenge (Study Day 100) in Group 2. There were no clinical signs of HSV-2 related disease observed in Groups 1, 3, 4 and 5.

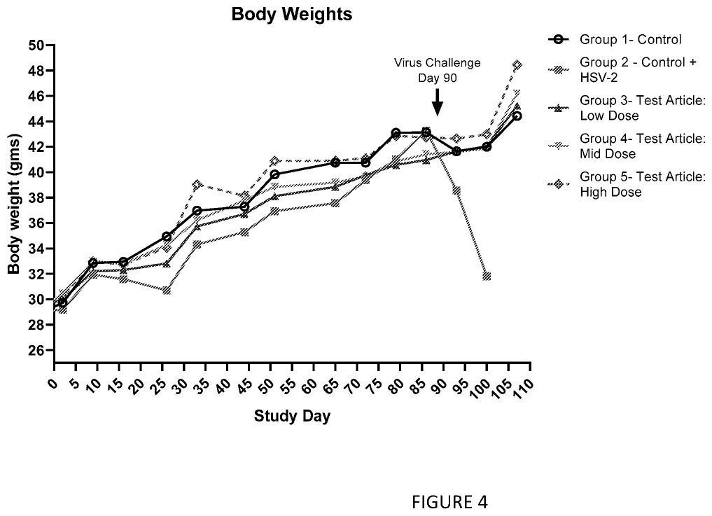

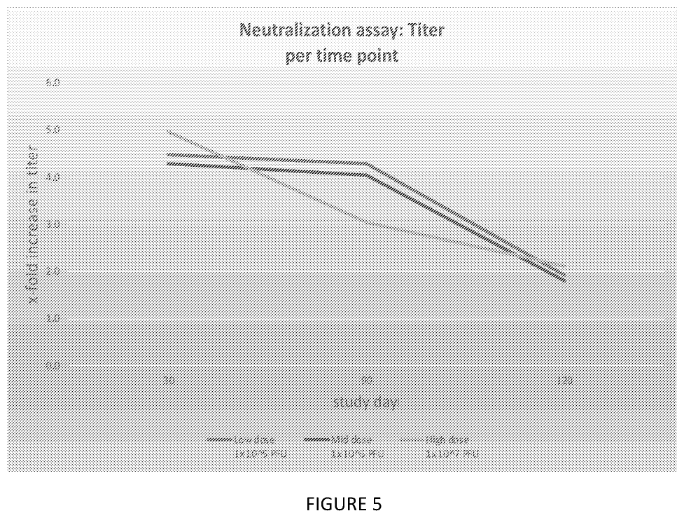

All animals accrued a cumulative weight gain over the course of the study following dosing (Day 0) to the time of Study Day 114. Weight loss was observed in several animals following the HSV-2 viral challenge. There was no statistically significant change in group body weights prior to HSV-2 challenge between in the control (Group 1) and test article (Groups 3, 4 and 5) animals as determined by 2-way ANOVA. Only non-immunized mice (Group 2) showed a decrease in weight post-challenge (FIG. 4) and exhibited clinical symptoms (not shown). The clinical findings are consistent with the neutralization assay results—all immunized groups (3-5) show a sustained increase in neutralizing antibody titer versus control (Group 1) (FIG. 5).

### CONCLUSION

Vaccinated animals in Group 3 (Test article low dose+HSV-2 challenge), Group 4 (Test article mid dose+HSV-2 challenge), and Group 5 (Test article High dose+HSV-2 challenge) were completely protected against viral challenge and did not show any clinical signs of infection (Clinical scores=0) or mortality over the post-challenge observation period. This study demonstrated the prophylactic efficacy of this HSV-2 vaccine.

What is claimed is:

1. A mutant herpesvirus comprising a modified RL2 gene sequence, wherein the modified RL2 gene sequence encodes a polypeptide comprising one or more fragments of infected cell protein 0 (ICP0), wherein the polypeptide comprises or consists of, from the N-terminus to the C-terminus:
a. a first ICP0 sequence comprising a sequence having at least 80% sequence identity to SEQ ID NO: 1 and comprising no more than 30 amino acids;
b. optionally, a first non-ICP0 sequence;
c. a second ICP0 sequence comprising a sequence having at least 80% sequence identity to SEQ ID NO: 2 and comprising no more than 30 amino acids; and,
d. optionally, a second non-ICP0 sequence:
wherein the first and second non-ICP0 sequences have less than 10% sequence identity to ICP0, and
wherein the mutant herpesvirus is substantially avirulent and immunogenic.

2. The mutant herpesvirus of claim 1, wherein the herpesvirus is selected from herpes simplex virus 1 (HSV-1) or herpes simplex virus 2 (HSV-2).

3. The mutant herpesvirus of claim 1 or claim 2, wherein the polypeptide does not comprise an ICP0 region selected from a RING finger domain, a nuclear localization signal (NLS) domain, and an oligomerization domain.

4. The mutant herpesvirus of any one of claims 1-3, wherein the polypeptide does not comprise the first non-ICP0 sequence or the second non-ICP0 sequence.

5. The mutant herpesvirus of claim 4, wherein the polypeptide has at least 80% sequence identity to SEQ ID NO: 7.

6. The mutant herpesvirus of any one of claims 1-3, wherein the polypeptide comprises the second non-ICP0 sequence and does not comprise the first non-ICP0 sequence.

7. The mutant herpesvirus of claim 6, wherein the polypeptide has at least 80% sequence identity to SEQ ID NO: 8.

8. The mutant herpes virus of any one of claims 1-3, wherein the polypeptide comprises the first non-ICP0 sequence and does not comprise the second non-ICP0 sequence.

9. The mutant herpesvirus of claim 8, wherein the polypeptide has at least 80% sequence identity to SEQ ID NO: 9.

10. The mutant herpesvirus of any one of claims 1-3, wherein the polypeptide comprises the first non-ICP0 sequence and the second non-ICP0 sequence.

11. The mutant herpesvirus of any one of claims 1-10, wherein the first non-ICP0 sequence comprises at least 200 amino acids.

12. The mutant herpesvirus of claim 11, wherein the first non-ICP0 sequence comprises or consists of a green florescent protein (GFP) sequence.

13. The mutant herpesvirus of claim 11, wherein the first non-ICP0 sequence has at least 80% sequence identity to SEQ ID NO: 3.

14. The mutant herpesvirus of any one of claims 1-13, wherein the second non-ICP0 sequence comprises at least 100 amino acids.

15. The mutant herpesvirus of claim 14, wherein the second non-ICP0 sequence has at least 80% sequence identity to SEQ ID NO: 4.

16. The mutant herpesvirus of any one of claims 1-15, wherein the polypeptide has at least 80% sequence identity to SEQ ID NO: 5.

17. An immunogenic composition comprising a pharmaceutically acceptable carrier and the mutant herpesvirus as of any one of claims 1-16.

18. A method of immunizing a subject, the method comprising administering to the subject the immunogenic composition of claim 17.

19. The method of claim 18, wherein the subject is human.

20. The method of claim 18 or claim 19, wherein the immunogenic composition is administered to the subject in a therapeutically effective amount to induce an immunological response to the mutant herpesvirus.

21. The method of any one of claims 18-20, further comprising administering to the subject a booster dose of the immunogenic composition of claim 10.

22. The method of and one of claims 18-21, for treating a herpesvirus infection in the subject.

23. The method of any one of claims 18-21, for preventing or inhibiting a herpesvirus infection in the subject.

24. The method of claim 22 or claim 23, wherein the herpesvirus infection is oral herpes.

25. The method of claim 23 or claim 23, wherein the herpesvirus infection is genital herpes.

26. A mutant ICP0 polypeptide comprising or consisting of, from the N-terminus to the C-terminus:
a. a first ICP0 sequence comprising a sequence having at least 80% sequence identity to SEQ ID NO: 1 and comprising no more than 30 amino acids;
b. optionally, a first non-ICP0 sequence;
c. a second ICP0 sequence comprising a sequence having at least 80% sequence identity to SEQ ID NO: 2 and comprising no more than 30 amino acids; and
d. optionally, a second non-ICP0 sequence:
wherein the first and second non-ICP0 sequences have less than 10% sequence identity to ICP0.

27. The mutant ICP0 polypeptide of claim 26, wherein the ICP0 is from herpesvirus is selected from herpes simplex virus 1 (HSV-1) or herpes simplex virus 2 (HSV-2).

28. The mutant ICP0 polypeptide of claim 26 or claim 27, wherein the polypeptide does not comprise an ICP0 region selected from a RING finger domain, a nuclear localization signal (NLS) domain, and an oligomerization domain.

29. The mutant ICP0 polypeptide of any one of claims 26-28, wherein the polypeptide does not comprise the first non-ICP0 sequence or the second non-ICP0 sequence.

30. The mutant ICP0 polypeptide of claim 29, wherein the polypeptide has at least 80% sequence identity to SEQ ID NO: 7.

31. The mutant ICP0 polypeptide of any one of claims 26-28, wherein the polypeptide comprises the second non-ICP0 sequence and does not comprise the first non-ICP0 sequence.

32. The mutant ICP0 polypeptide of claim 31, wherein the polypeptide has at least 80% sequence identity to SEQ ID NO: 8.

33. The mutant ICP0 polypeptide virus of any one of claims 26-28, wherein the polypeptide comprises the first non-ICP0 sequence and does not comprise the second non-ICP0 sequence.

34. The mutant ICP0 polypeptide of claim 33, wherein the polypeptide has at least 80% sequence identity to SEQ ID NO: 9.

35. The mutant ICP0 polypeptide of any one of claims 26-28, wherein the polypeptide comprises the first non-ICP0 sequence and the second non-ICP0 sequence.

36. The mutant ICP0 polypeptide of any one of claims 26-35, wherein the first non-ICP0 sequence comprises at least 200 amino acids.

37. The mutant ICP0 polypeptide of claim 36, wherein the first non-ICP0 sequence comprises or consists of a green florescent protein (GFP) sequence.

38. The mutant ICP0 polypeptide of claim 36, wherein the first non-ICP0 sequence has at least 80% sequence identity to SEQ ID NO: 3.

39. The mutant ICP0 polypeptide of any one of claims 26-38, wherein the second non-ICP0 sequence comprises at least 100 amino acids.

40. The mutant ICP0 polypeptide of claim 39, wherein the second non-ICP0 sequence has at least 80% sequence identity to SEQ ID NO: 4.

41. The mutant ICP0 polypeptide of any one of claims 26-40, wherein the polypeptide has at least 80% sequence identity to SEQ ID NO: 5.

42. A polynucleotide comprising a sequence encoding the mutant ICP0 polypeptide of any one of claims 26-41.

43. A cell comprising the mutant herpesvirus of any one of claims 1-16.

44. A cell comprising the polynucleotide of claim 42.


SEQUENCE LISTING (11 sequences)

SEQ ID NO: 1 - protein, 18 aa, Human alphaherpesvirus 2
MEPRPGTSSRADPGPERP

SEQ ID NO: 2 - protein, 19 aa, Human alphaherpesvirus 2
GSCGGGPVGEEEAEAGGGG

SEQ ID NO: 3 - protein, 236 aa, Artificial Sequence
  Synthetic green fluorescent protein
LSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDEL

SEQ ID NO: 4 - protein, 124 aa, Artificial Sequence
  Synthetic construct
RRVRRVHGRDRPAPALPEFSLPAPLLHPVHEDLDSVAQHVSPVQHPGGVPDSGRDRQRVVQHHPDSERPPDPRGGRGGRAVRHGRGLYLDGQPADGPALPVAGGTHGPRPVAHPPVARHGRRGR

SEQ ID NO: 5 - protein, 397 aa, Artificial Sequence
  Synthetic construct
MEPRPGTSSRADPGPERPLSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELGSCGGGPVGEEEAEAGGGGRRVRRVHGRDRPAPALPEFSLPAPLLHPVHEDLDSVAQHVSPVQHPGGVPDSGRDRQRVVQHHPDSERPPDPRGGRGGRAVRHGRGLYLDGQPADGPALPVAGGTHGPRPVAHPPVARHGRRGR

SEQ ID NO: 6 - DNA, 1194 bp, Artificial Sequence
  Synthetic construct
atggaaccccggcccggcacgagctcccgggcggaccccggccccgagcggccgctgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgggatcctgcgggggtgggcccgtgggtgaggaggaagcggaagcgggagggggggggcgacgtgtgcgccgtgtgcacggacgagatcgccccgcccctgcgctgccagagttttccctgcctgcaccccttctgcatcccgtgcatgaagacctggattccgttgcgcaacacgtgtcccctgtgcaacaccccggtggcgtacctgatagtgggcgtgaccgccagcgggtcgttcagcaccatcccgatagtgaacgacccccggacccgcgtggaggccgaggcggccgtgcggtccggcacggccgtggactttatctggacgggcaacccgcggacggccccgcgctccctgtcgctggggggacacacggtccgcgccctgtcgcccacccccccgtggcccggcacggacgacgaggacgatga

SEQ ID NO: 7 - protein, 37 aa, Artificial Sequence
  Synthetic construct
MEPRPGTSSRADPGPERPGSCGGGPVGEEEAEAGGGG

SEQ ID NO: 8 - protein, 161 aa, Artificial Sequence
  Synthetic construct
MEPRPGTSSRADPGPERPGSCGGGPVGEEEAEAGGGGRRVRRVHGRDRPAPALPEFSLPAPLLHPVHEDLDSVAQHVSPVQHPGGVPDSGRDRQRVVQHHPDSERPPDPRGGRGGRAVRHGRGLYLDGQPADGPALPVAGGTHGPRPVAHPPVARHGRRGR

SEQ ID NO: 9 - protein, 273 aa, Artificial Sequence
  Synthetic construct
MEPRPGTSSRADPGPERPLSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELGSCGGGPVGEEEAEAGGGG

SEQ ID NO: 10 - protein, 397 aa, Artificial Sequence; identical to SEQ ID NO: 5
  Synthetic construct

SEQ ID NO: 11 - protein, 801 aa, Human alphaherpesvirus 2 strain MS
MEPRPGTSSRADPGPERPPRQTPGTQPAAPHAWGMLNDMQWLASSDSEEETEVGISDDDLHRDSTSEAGSTDTEMFEAGLMDAATPPARPPAERQGSPTPADAQGSCGGGPVGEEEAEAGGGGDVCAVCTDEIAPPLRCQSFPCLHPFCIPCMKTWIPLRNTCPLCNTPVAYLIVGVTASGSFSTIPIVNDPRTRVEAEAAVRSGTAVDFIWTGNPRTAPRSLSLGGHTVRALSPTPPWPGTDDEDDDLADADYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFFSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAARPRKRRGSDSGPAASSSASSSAAPRSPLAPQGVGAKRAAPRRAPDSDSGDRGHGPLAPASAGAAPPSASPSSQAAVAAAASSSSSSSSSASSSAGGAGGSVASASGAGERRETSLGPRAAAPRGPRKCARKTRHAEGGPEPGARDPAPGLTRYLPIAGASSVVALAPYVNKTVTGDCLPILDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE